(19) United States
(12) Patent Application Publication
(10) Pub. No.: US 2026/0083896 A1
(43) Pub. Date: Mar. 26, 2026

(54) ERGONOMICAL MEDICAL DEVICE HANDLE WITH FLUID COUPLING

(71) Applicant: Baylis Medical Technologies Inc., Mississauga (CA)

(72) Inventors: Patrick Ryan, Toronto (CA); Amanda Centazzo-Colella, Bowmanville (CA); Amanda Hartley, Caledon (CA); Michael Dallimore, Barrie (CA)

(21) Appl. No.: 19/403,145

(22) Filed: Nov. 27, 2025

Related U.S. Application Data
(63) Continuation-in-part of application No. PCT/IB2024/055349, filed on May 31, 2024.
(60) Provisional application No. 63/505,463, filed on Jun. 1, 2023.

(51) Int. Cl. A61M 1/00; A61M 5/00

(52) U.S. Cl. CPC A61M 1/87; A61M 5/007; A61M 2205/3344

(57) ABSTRACT

A method and apparatus are disclosed for improving ergonomics of the proximal end of a medical device used to perform a Transjugular Intrahepatic Portosystemic Shunt (TIPS) procedure. The present invention provides a telescoping assembly comprising a shared flush port for use through a lumen of multiple devices. The present invention further provides locking features for advancing devices in a controlled manner, while being able to securely hold the other devices.

### TECHNICAL FIELD

The disclosure relates to medical devices, and more particularly to for coupling medical devices and manipulating fluids through coupled medical devices, specifically during an intrahepatic portosystemic shunt procedure.

### BACKGROUND OF THE ART

For patients suffering from portal hypertension, a Transjugular Intrahepatic Portosystemic Shunt (TIPS) procedure may be performed. In this procedure, a shunt is created from the hepatic vein to the portal vein, which allows blood flow to bypass the liver and alleviates the portal pressure. Creation of the shunt is done percutaneously and uses various sheaths, catheters, and wires.

Existing puncture kits for use in a TIPS procedure require numerous exchanges, i.e., inserting, removing, and reinserting multiple devices within a patient. In some cases, the same device is inserted and removed multiple times. The ability to use one device for multiple steps can reduce the number of exchanges, thereby reducing procedure time and decreasing procedural complexity.

Ergonomic and functionality problems can arise when multiple devices need to be handled simultaneously by a single operator (physician). Typically, during a TIPS procedure, multiple elongate devices are used in telescoped arrangement, for example, a sheath, a dilator, a catheter, a needle, and a guidewire. A physician may have difficulty manipulating one device with one hand, while maintaining control of the other devices with the other hand.

Another difficulty is the ability to manipulate multiple flush ports on each device's proximal end for aspirating, injecting contrast, and measuring pressure. The ability to handle the back end of the system can become difficult due to the number of external devices connected to each flush port.

### BRIEF DESCRIPTION OF THE DRAWINGS

In order that the invention may be readily understood, embodiments of the invention are illustrated by way of examples in the accompanying drawings, in which:

FIG. 1 is an illustration of the anatomy of a liver and surrounding blood vessels;

FIGS. 2A-2B are illustrations of a telescoping assembly for performing a TIPS procedure in accordance with an embodiment of the present invention;

FIG. 3A is an illustration of a steerable sheath in accordance with an embodiment of the present invention;

FIG. 3B is a cross-sectional illustration of the steerable sheath of FIG. 3A in accordance with an embodiment of the present invention;

FIG. 4A is an illustration of a dilator in accordance with an embodiment of the present invention;

FIG. 4B is a cross-sectional illustration of the dilator of FIG. 4A in accordance with an embodiment of the present invention;

FIG. 4C is an illustration of a dilator in accordance with an alternative embodiment of the present invention;

FIG. 4D is a cross-sectional illustration of the dilator of FIG. 4C in accordance with an alternative embodiment of the present invention;

FIG. 4E is an illustration of a dilator in accordance with a further alternative embodiment of the present invention;

FIG. 5A is a cross-sectional illustration of a dilator received within a sheath in accordance with an embodiment of the present invention;

FIG. 5B is a cross-sectional illustration of a dilator received within a sheath in accordance with an alternative embodiment of the present invention;

FIG. 5C is a cross-sectional illustration of a dilator received within a sheath in accordance with a further alternative embodiment of the present invention;

FIG. 5D is a cross-sectional illustration of a dilator received within a sheath in accordance with a further alternative embodiment of the present invention;

FIGS. 6A-6C are illustrations of alignment features in accordance with an embodiment of the present invention;

FIGS. 6D and 6E is an illustration of alignment features in accordance with an alternative embodiment of the present invention;

FIGS. 6F-6I are cross-sectional illustrations of the alignment features of FIG. 6D in accordance with an alternative embodiment of the present invention;

FIGS. 7A-7F are illustrations of locking features in accordance with a further alternative embodiment of the present invention;

FIGS. 7G-7H are cross-sectional illustrations of an angled catheter received within a dilator;

FIG. 8A is an illustration of a dilator comprising a side port in accordance with an embodiment of the present invention;

FIG. 8B is a cross-sectional illustration of the dilator comprising a side port of FIG. 8A in accordance with an embodiment of the present invention;

FIG. 8C is an illustration of a side port in accordance with an alternative embodiment of the present invention;

FIG. 9A is an illustration of a catheter comprising an elongate aperture in accordance with an embodiment of the present invention;

FIG. 9B is a cross-sectional illustration of the catheter comprising an elongate aperture of FIG. 9A in accordance with an embodiment of the present invention;

FIG. 9C is an illustration of a catheter comprising a stiff proximal region in accordance with an embodiment of the present invention;

FIG. 9D is a cross-sectional illustration of the catheter comprising a stiff proximal region of FIG. 9C in accordance with an embodiment of the present invention;

FIG. 10A is an illustration of a wire received within a catheter comprising an elongate aperture in accordance with an embodiment of the present invention;

FIG. 10B is a cross-sectional illustration of FIG. 10A;

FIG. 11 is a cross-sectional illustration of a wire, a catheter comprising an elongate aperture, and a dilator comprising a side port, in accordance with an embodiment of the present invention;

FIG. 12A is an illustration of an advancement mechanism for advancing a device in accordance with an embodiment of the present invention;

FIG. 12B(i)-12B(ii) is an illustrations of an advancement mechanism for advancing a device in accordance with an alternative embodiment of the present invention;

FIG. 12C(i)-12C(iv) are illustrations of an advancement mechanism for advancing a device in accordance with a further alternative embodiment of the present invention;

FIG. 13 is a flow diagram showing a method for injecting contrast fluid during a TIPS procedure in accordance with an embodiment of the present invention;

FIGS. 14A-14B are illustrations of the assembly in use during the method of FIG. 13 in accordance with an embodiment of the present invention;

FIG. 15 is a flow diagram showing a method for advancing devices during a TIPS procedure in accordance with an embodiment of the present invention;

FIG. 16A(i)-16B(ii) are illustrations of the assembly in use during the method of FIG. 15 in accordance with an embodiment of the present invention; and

FIG. 17 is a flow diagram showing an alternative method for advancing devices during a TIPS procedure in accordance with an embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

In one broad aspect, embodiments of the present invention comprise a telescoping assembly comprising: a first elongate device, the first elongate device comprising a first device proximal region and defining a first device distal opening, the first elongate device further defining a first device lumen terminating at the first device distal opening, and a first device side opening in the first device proximal region in communication with the first device lumen; and a second elongate device configured to be insertable into the first device lumen and moveable relative thereto, the second elongate device comprising a second device proximal region and defining a second device distal opening, the second elongate device further defining a second device lumen terminating at the second device distal opening, and a second device side opening in the second device proximal region in communication with the second device lumen; the second elongate device is insertable into the first device lumen to an aligned configuration, whereby the first device side opening and the second device side opening are aligned to provide a passage from an external environment to the second device lumen through the first and second device side openings.

As a feature of this aspect, at least one of the first device and second device comprises an alignment feature, for confirming when the first device and second device are in the aligned configuration.

As a feature of this aspect, at least one of the first device and second device comprises a guiding feature for facilitating arrangement of the devices into the aligned configuration.

As a feature of this aspect, the alignment feature comprises a longitudinal alignment feature.

As a feature of this aspect, the alignment feature comprises a radial alignment feature.

As a feature of this aspect, at least one of the first device and second device comprises a sealing feature for creating a seal between the first device side opening and the second device side opening.

As a feature of this aspect, at least one of the first device and second device comprises a locking feature, whereby when the telescoping assembly is in a locked state, movement between the two is limited, and the locking feature is configured to allow for transition between the locked state and an unlocked state.

As a feature of this aspect, in the locked state, the first and second devices are in the aligned configuration.

As a feature of this aspect, the second device lumen comprises a secondary lumen and the second elongate device further defines a distal end opening and a primary lumen terminating at the distal end opening, the primary lumen having a larger diameter than the secondary lumen for receiving a third elongate device.

In another broad aspect, embodiments of the present invention comprise a method for interacting with fluid using a telescoping assembly, the telescoping assembly comprising a first elongate device and a second elongate device configured to be insertable into the first elongate device and moveable relative thereto, the first elongate device defining a first side opening and the second elongate device defining a second side opening, the method comprising the steps of: interacting with fluid located in a lumen of the second elongate device through the first side opening; withdrawing the second elongate device from the first elongate device; and interacting with fluid located in a lumen of the first elongate device through the first side opening.

As a feature of this aspect, at least one of the steps of interacting with fluid located in a lumen of the second elongate device, or the step of interacting with fluid located in a lumen of the first elongate device, is selected from the group consisting of: aspirating, injecting contrast, and measuring a pressure.

As a feature of this aspect, the method further comprises a step of inserting the second elongate device into the first elongate device.

As a feature of this aspect, the method further comprises a step of aligning the second side opening with the first side opening.

As a feature of this aspect, the method further comprises a step of securing the second device to the first device.

In another broad aspect, embodiments of the present invention comprise: a medical apparatus comprising: a first elongate device comprising a first device proximal region and a first device distal region, the first elongate device defining a first device distal opening in the first device distal region and a first device proximal opening in the first device proximal region and a first device lumen extending therebetween; a side port associated with the first device proximal region; a second elongate device configured to be insertable into the first device lumen and moveable relative thereto, the second elongate device comprising a second device proximal region and a second device distal region, the second elongate device defining a second device distal opening in the second device distal region and a second device proximal opening in the second device proximal region and a second device lumen extending therebetween, the second device further comprising a sidewall in the second device proximal region, and further defining an elongated aperture in the sidewall; and a third elongate device, configured to be insertable into the second lumen and moveable therethrough; the side port and elongated aperture are configured such that, when the first and second devices are in an aligned configuration, the third device is insertable through both the side port and elongated aperture into the second device lumen.

As a feature of this aspect, while the third elongate device is positioned through the elongated aperture, longitudinal movement of the second elongate device relative to the first elongate device is limited substantially to a length of the elongate aperture.

As a feature of this aspect, the medical apparatus further comprises at least one locking feature, associated with the first elongate device, for restraining movement of the third elongate device relative to the first elongate device.

As a feature of this aspect, the first elongate device defines the side port.

As a feature of this aspect, the medical apparatus further comprises a multi-channel attachment connected to the first proximal opening, the multi-channel attachment defining the side port.

As a feature of this aspect, the medical apparatus further comprises a fourth device, the fourth device comprising a locking feature for prohibiting movement of the first device relative to the fourth device.

For explanatory purposes, the systems and methods disclosed herein are generally described with reference to a TIPS procedure, where a tract is created from the hepatic vein to the portal vein. As would be apparent to one skilled in the art, the systems and methods described herein are also applicable to a direct intrahepatic shunt (DIPS) procedure. Certain aspects of this disclosure are also applicable to other medical procedures as well beyond TIPS and DIPS.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

A method of performing a TIPS procedure using a telescoping assembly is disclosed in U.S. Pat. No. 11,324,548 B2, granted on May 10, 2022 to Baylis Medical. The method involves performing part of a TIPS procedure using a telescoped assembly comprising a flexible radiofrequency (RF) device, a catheter, a dilator, and a steerable sheath.

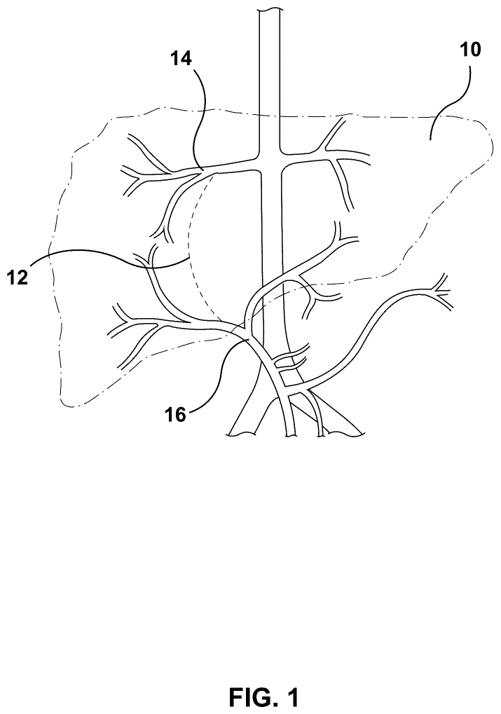

Referring to FIG. 1, a diagram of a liver 10 and adjoining structures is shown. FIG. 1 shows a dashed line 12 representing a path for a tract to be created between the hepatic vein 14 and portal vein 16, for example during a TIPS procedure.

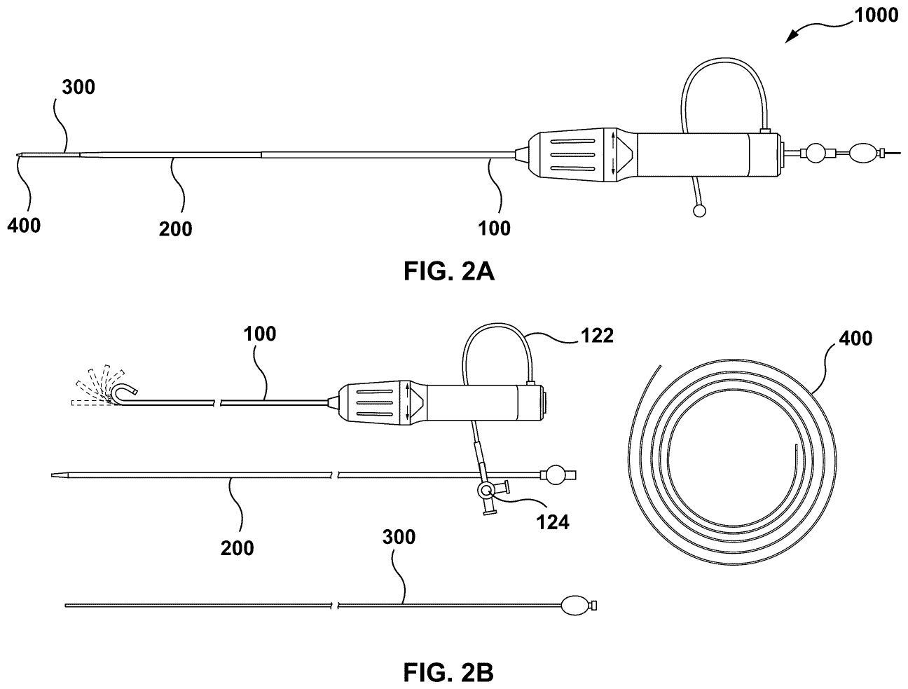

Referring to FIGS. 2A to 2B, according to one embodiment of the present invention, the telescoping assembly 1000 for performing at least part of a TIPS procedure comprises a sheath 100 (which may also be referred to as a steerable sheath), a dilator 200 (which may also be referred to as a flexible dilator), a catheter 300 (which may also be referred to as a microcatheter or crossing catheter), and a puncture wire 400.

In one embodiment, puncture wire 400 is a wire that delivers radiofrequency energy to create a puncture through the liver. In other embodiments, puncture wire 400 is a needle, a stylet, a mechanical wire, or other guidewire that does not deliver RF energy. In some embodiments, puncture wire 400 may also be used as guidewire. In some embodiments a separate guidewire or rail wire may be swapped with puncture wire 400 after the puncture has been performed. In one specific example, puncture wire 400 is a mechanical needle made of stainless steel and has an outside diameter of approximately 0.035″. In some embodiments, puncture wire 400 may be capable of producing a puncture during a TIPS procedure and also capable of acting as a rail or guidewire during other parts of a TIPS procedure.

In one specific example, telescoping assembly 1000 comprises a 10 French (Fr) steerable sheath, a 10 Fr flexible dilator, a 5 Fr crossing catheter, and a 0.035″ RF guidewire. In one example, puncture wire 400 is of the type disclosed in U.S. Pat. No. 9,510,900 B2, granted Dec. 6, 2016, to Baylis Medical Company Inc.

In such an example, the assembly 1000 includes a sheath 100, a dilator 200 received within the sheath 100, a catheter 300 received within the dilator 200, and a puncture wire 400 received within the catheter 300, each component being received in a telescoping arrangement relative to the others. FIG. 2A shows assembly 1000 with all four devices in a telescoped arrangement and FIG. 2B shows each device separate and removed from the others.

In one embodiment of the present invention, sheath 100 comprises a sealed access port on the handle, described herein, that connects to valve 124, via a flexible hose 122. Valve 124 may be stopcock, check valve, dual check valve, or any other device that connects to other external fluid manipulation devices (not shown). In some examples, valve 124 connects to external devices via a luer-lock or standard luer fittings.

One or more fluid manipulation devices can interact with fluids through the device lumens. In one example, a fluid manipulation device is an injection device, such as a syringe, which may be used to aspirate or inject fluids, for example contrast fluid. In another example, the fluid manipulation device is a power injector, which is used to inject contrast fluid at a controlled rate. In another example, the fluid manipulation device is a pressure transducer, which may be used to measure pressure within a blood vessel.

For descriptive purposes, any of the sheath, dilator, catheter, or puncture wire may be referred to as a “device” or “elongate device”, and the same modifications and relationships between the sheath and dilator may apply between the dilator and catheter, where applicable. Devices may also be referred to as either an “inner device” or an “outer device”. For example, when describing the catheter and the puncture wire, the puncture wire may be positioned within the catheter, such that the catheter would be referred to as the outer device and the puncture wire would be referred to as the inner device.

In one embodiment, each device of telescoping assembly 1000 is separate and fully removable from all other devices. In other words, the devices may be assembled in any combination at any one time. If more space is needed within a lumen of one device, for example the sheath, then one or more devices, for example the dilator and catheter, can be removed to create space in the sheath lumen. Further, each device may be “backloadable”, i.e., an inner device, may be removable from an outer device without having to remove a different inner device. For example, the dilator 200 may be removable from the sheath 100 without first having to remove the catheter 300.

Having each device fully removeable from the assembly could offer advantages. For example, if a problem occurs with any one device, it could be exchanged for a new device. This exchange could be made at any point during a TIPS procedure.

### Flush Ports

At various times during a TIPS procedure, an operator may need to aspirate, measure pressure, or inject contrast fluid in multiple blood vessels. Performing these functions requires at least one fluid manipulation device coupled to the proximal end of at least one device in the assembly, via a flush port. Each of the sheath, dilator, or catheter may have a flush port connected to a fluid manipulation device. The flush port may be defined by the body (shaft) of the device, or may be a separate element, for example a hub, connected to the proximal end of the device.

One example of manipulating fluids during a TIPS procedure involves injecting contrast fluid into the hepatic vein 14 before a puncture is made through the liver 10. Contrast fluid may be used to visualize the hepatic vein 14 and other blood vessels using known imaging modalities. Prior to the puncture, the dilator 200 may be inside of the sheath 100, and contrast fluid would have to be injected through a lumen in the dilator 200.

At a later point in the procedure, after the puncture has been made and the catheter 300 and puncture wire 400 are advanced through the parenchyma tract into the portal vein 16, the sheath 100 remains in the hepatic vein 14. In order to visualize the hepatic vein 14 and parenchyma tract, contrast fluid would need to be injected again. At this point in the procedure, the dilator 200 may have been removed from the assembly, so contrast fluid would have to be injected through the sheath 100 lumen. Current techniques for injecting contrast at these two stages require at least two flush ports and at least two fluid manipulation devices, one for the dilator 200 and one for the sheath 100.

A single operator may have difficulty handing multiple flush ports in addition to the assembled devices. One option to reduce the number of flush ports is to use only the sheath 100 for both steps. However, this requires removing the dilator 200 every time the sheath 100 flush port is used, and then potentially reinserting the dilator 200 if and when needed. Doing this prolongs the procedure and adds complexity. The ability to manipulate fluid through multiple devices without having to remove and reinsert them could be advantageous.

The present inventors have conceived of novel and inventive devices, assemblies, and methods for improving the ergonomics and handleability of the devices in TIPS procedures, while also reducing the number of exchanges. More specifically, the present invention reduces the number of flush ports on the proximal end and allows for manipulating fluids through two devices, using a single flush port, without the need to remove the inner device.

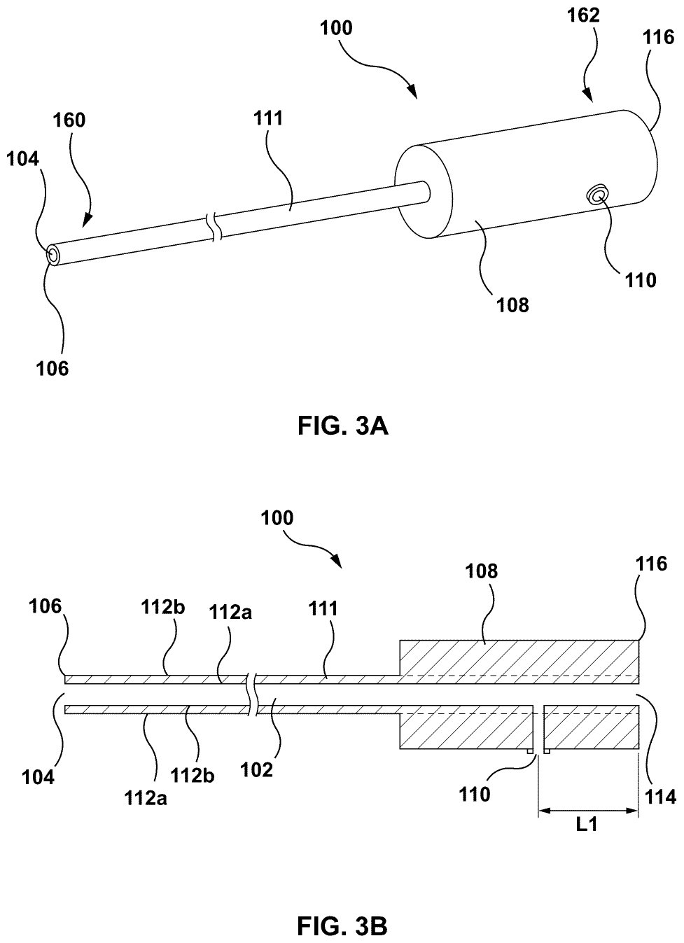

Referring now to FIGS. 3A and 3B, in one embodiment of the present invention, sheath 100 comprises a shaft 111 having an inner wall 112a and outer wall 112b defining a primary lumen 102 for receiving another device, for example dilator 200. Primary lumen 102 extends between a distal opening 104 at distal end 106 and proximal opening 114 at proximal end 116. Dilator 200 is configured to be insertable into sheath primary lumen 102 and moveable in a longitudinal direction relative to sheath 100. In one embodiment, sheath 100 comprises a hemostatic valve (not shown) at the proximal end 116 to allow other devices to enter lumen 102, and to prevent fluids from escaping through proximal opening 114.

Sheath 100 further comprises a distal region 160 and proximal region 162, and further defines a side opening or side port 110, located in the proximal region 162. In one embodiment, sheath 100 comprises handle 108, located in the proximal region, and handle 108 defines side port 110. In one example, side port 110 may be located a distance L1 from the proximal end 116. In one example, L1 is between about 5 mm and about 15 mm. A proximal end of shaft 111 is coupled to handle 108. In one embodiment, handle comprises one or more actuators (not) shown for deflecting distal region 160, allowing shaft 111 to adopt one or more curved configurations.

Side port 110 extends between inner wall 112a, outer wall 112b, and handle 108, and is in fluid communication with primary lumen 102, such that fluids can travel from distal opening 104, through primary lumen 102, through side port 110, to an external environment, i.e., outside of the sheath. Fluids can travel in either direction. In other words, there is a passage for fluids to flow from the distal opening 104 to side port 110.

Side port 110 fluidically connects to one or more devices in an external environment, i.e., devices separate from assembly 1000. In use, in one example, an external device is fluidically connected to lumen 102, via side port 110 through flexible hose 122 and valve 124. Side port 110, flexible hose 122 and valve 124 may collectively be referred to as “flush port” or “injection port”. In another example, valve 124 connects directly to side port 110. For clarity, the term fluidically connected means a connection where fluids may flow, e.g., if A is fluidically connected to B, there is a path for fluids to travel from A to B and from B to A.

Side port 110 is in fluid communication with a side opening in dilator 200, described herein, so the two devices can share a single flush port.

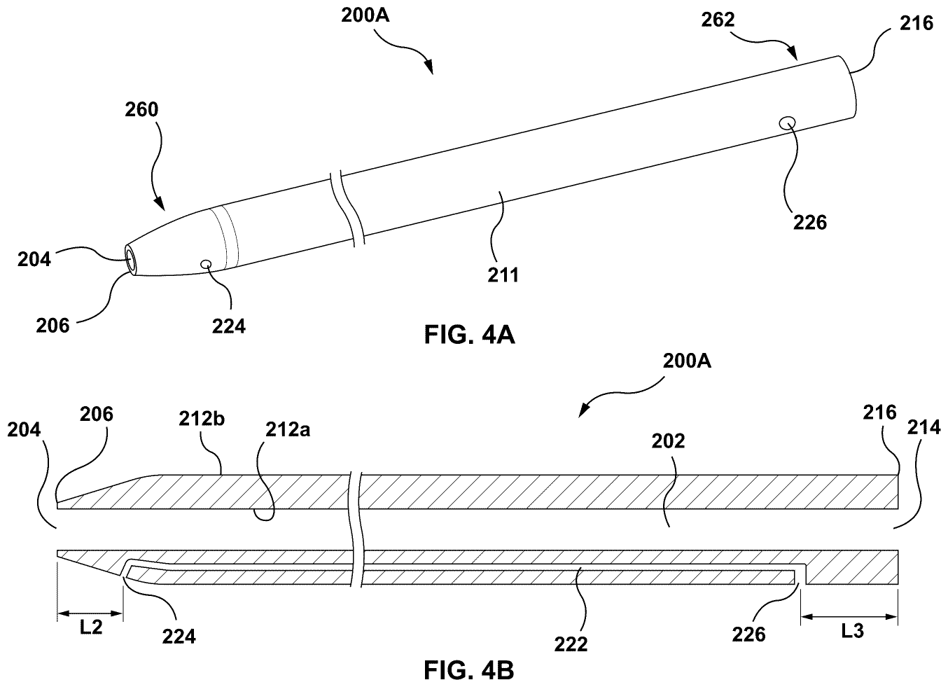

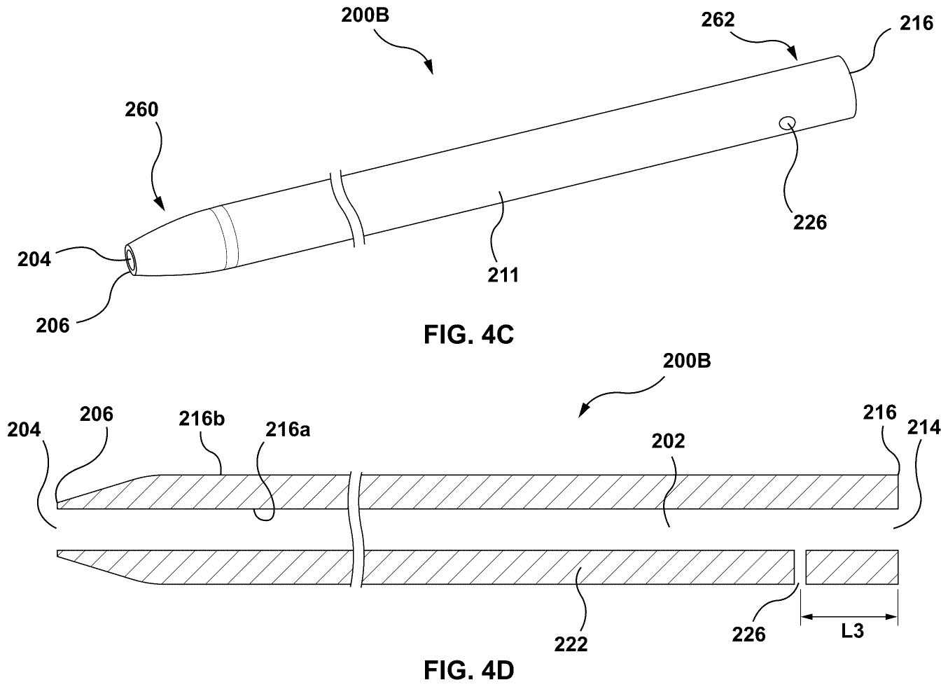

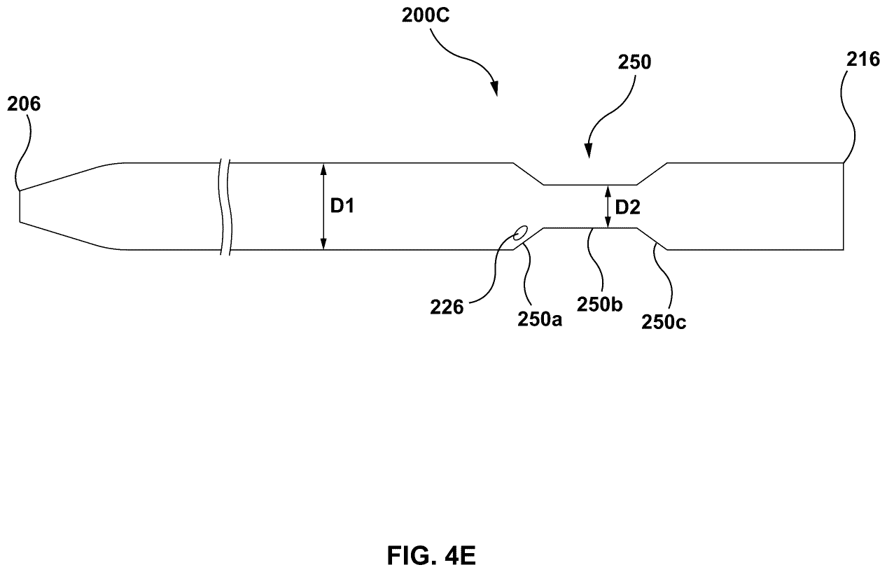

Referring to FIGS. 4A and 4B, in one embodiment of the present invention, dilator 200A comprises an elongate member 211 having an inner wall 212a and outer wall 212b, which define a primary lumen 202. Primary lumen 202 extends between distal opening 204 at distal end 206 and proximal opening 214, at proximal end 216. Primary lumen 202 has an inner diameter large enough to receive a third device, for example catheter 300 or puncture wire 400.

In one embodiment, inner wall 212a and outer wall 212b further define a secondary lumen 222 that extends from secondary distal opening 224 and secondary proximal opening 226. Secondary lumen 222 is located between inner wall 212a and outer wall 212b and has a smaller diameter than primary lumen 202. In some embodiments, secondary distal opening 224 and secondary proximal opening 226 are side apertures or side openings, defined by the outer wall 212b.

In one embodiment, secondary distal opening 224 is a side opening and is located at a distance L2 from the distal end 206. Secondary proximal opening 226 is a side opening and is located at a distance L3 from the proximal end 216. In one specific example L2 is between about 5 mm and about 10 mm, and L3 is between about 5 mm and about 15 mm. Secondary proximal opening 226 is in fluid communication with secondary distal opening 224, via secondary lumen 222. In other words, there is a passage for fluids to travel through secondary lumen 222. Fluids may travel in either direction through secondary lumen 222, i.e., fluids may enter or exit either of secondary distal opening 224 or secondary proximal opening 226. In such an embodiment, secondary lumen 222 may be used to manipulate fluids while catheter 300 is inserted within dilator primary lumen 202.

One or both of sheath 100 or dilator 200A may include one or more sealing features (not shown), capable of creating a fluid-tight seal between secondary proximal opening 226 and side port 110, such that no fluid escapes secondary lumen 222 except through the side port 110. In other words, no fluids will exit secondary proximal opening 226 and enter sheath primary lumen 102, all fluid that enters secondary distal opening 224 will only exit through side port 110 when sheath 100 and dilator 200A are in an aligned configuration. Sealing features can be, for example, a gasket, a hemostatic valve, or other similar components.

For clarity, an “aligned configuration” means sheath 100 and dilator 200A are positioned in longitudinal and rotational directions with respect to each other, such that side port 110 and secondary proximal opening 226 overlap, or are configured in some other way, such that fluids can flow through side port 110 and secondary proximal opening 226, in either direction. In other words, there is a passage from an external environment, through the sheath side port 110 to a lumen of the dilator 200, for example secondary lumen 222. Conversely, “unaligned” and “non-aligned” are used to mean not in an aligned configuration.

In another embodiment, as shown in FIGS. 4C and 4D, dilator 200B defines secondary proximal opening 226 that is in fluid communication with dilator primary lumen 202. In such an embodiment, primary lumen 202 is used to manipulate fluids through dilator distal opening 204. In other words, dilator 200B does not define a secondary lumen, and the passage extends from secondary proximal opening 226 to distal opening 204. When dilator 200B is inserted into sheath 100, and is in an aligned configuration, there is a path for fluids to travel through sheath side port 110 to dilator primary lumen 202 and exit dilator distal opening 204. In other words, there is a passage from an external environment, through the sheath side port 110 to the dilator primary lumen 202.

Wither reference to FIG. 4E, in another embodiment, dilator 200C defines a reduced diameter portion 250, and a secondary proximal opening 226 is located within the reduced diameter portion 250. In one embodiment, secondary proximal opening 226 is fluidically connected to secondary distal opening 224 (not shown) through secondary lumen 222 (not shown), in a configuration similar to dilator 200A, described herein. In a further embodiment, secondary proximal opening 226 is fluidically connected to distal opening 204 through primary lumen 202, in a configuration similar to dilator 200B.

In one specific example, reduced diameter portion 250 is substantially an hour-glass shape, defining a distal taper 250a, a reduced diameter segment 250b, and a proximal taper 250c. Secondary proximal opening 226 can be located in any one of distal taper 250a, reduced diameter segment 250b, or proximal taper 250c. The outer diameter D2 of reduced diameter segment 250b is less than the outer diameter D1 of dilator 200C. In one specific example, D2 is approximately 2.67 mm (8 Fr) and D1 is approximately 3.33 mm (10Fr). Reduced diameter portion 250 may have dimensions such that longitudinal movement of catheter 300 within dilator primary lumen 202 is not inhibited.

In such an embodiment, an aligned configuration is achieved when side port 110 is aligned with reduced diameter portion 250 in a longitudinal direction only. Such an embodiment does not require rotational alignment. In other words when side port 110 is longitudinally aligned with reduced diameter portion 250, an aligned configuration is achieved regardless of the rotation of sheath 100 or dilator 200 with respect to each other.

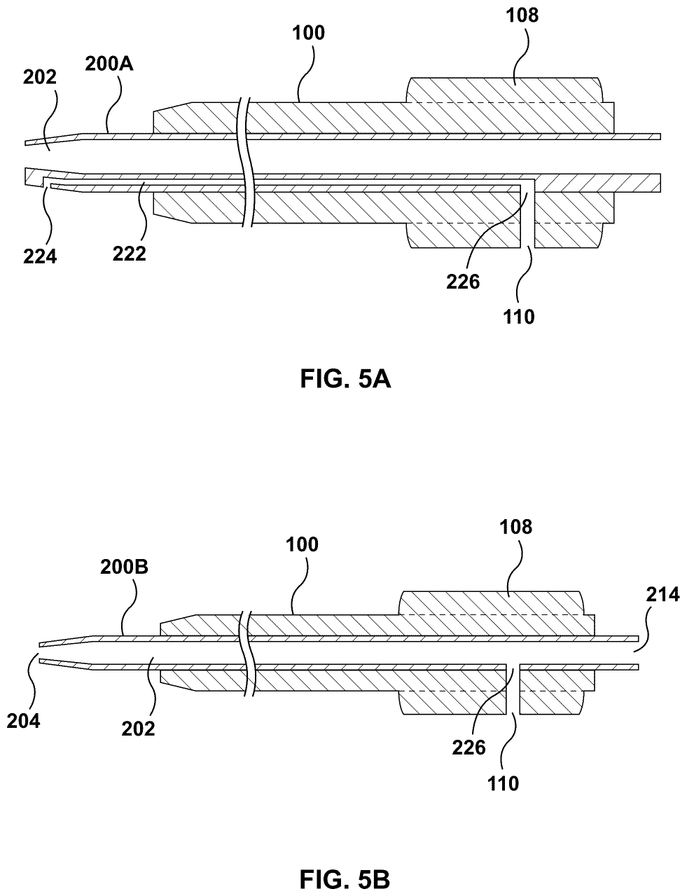

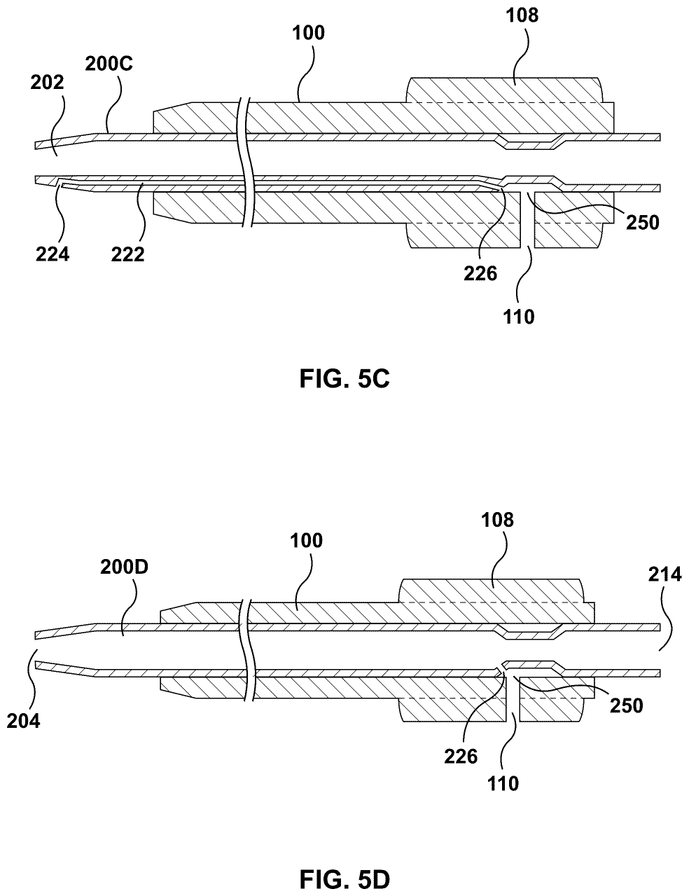

FIGS. 5A to 5D are side view cross-sectional illustrations showing various dilator-sheath combinations.

FIG. 5A shows dilator 200A received within sheath primary lumen 102, with sheath 100 and dilator 200A in an aligned configuration. Dilator 200A extends through sheath distal opening 104 and sheath proximal opening 114, and dilator secondary proximal opening 226 is aligned with sheath side port 110. An aligned configuration corresponds to a longitudinal alignment along a longitudinal axis and a rotational alignment about a longitudinal axis. In other words, the passage for manipulating fluids extends from dilator secondary distal opening 224, through secondary lumen 222, through secondary proximal opening 226, and through side port 110.

FIG. 5B shows dilator 200B received within sheath primary lumen 102, with both devices in an aligned configuration. The passage for manipulating fluids extends from dilator distal opening 204, through dilator primary lumen 202, through secondary proximal opening 226, and through side port 110.

FIG. 5C shows dilator 200C received withing sheath primary lumen 102 when both devices are in an aligned configuration. Dilator 200C comprises a secondary lumen 222 and reduced diameter portion 250. The aligned configuration comprises a longitudinal alignment only, as described herein. The passage for manipulating fluids extends from dilator secondary distal opening 224, through secondary lumen 222, through secondary proximal opening 226 into the reduced diameter portion 250, and through side port 110.

FIG. 5D shows dilator 200D is received with sheath primary lumen 102 and both devices are in an aligned configuration. Dilator 200D comprises a primary lumen 202 and reduced diameter portion 250. The aligned configuration comprises a longitudinal alignment only, as described herein. The passage for manipulating fluids extends from dilator distal opening 204, through primary lumen 202, through secondary proximal opening 226 into the reduced diameter portion 250, and through side port 110.

While in an aligned configuration, side port 110 is fluidically connected to an opening in the distal region of dilator 200, either dilator distal opening 204 or dilator secondary distal opening 224. While in an aligned configuration, an external device may be used to manipulate fluids, for example, injecting contrast fluid or measuring pressure at or near the distal opening. The external device is in fluid communication with valve 124, which is in fluid communication with side port 110.

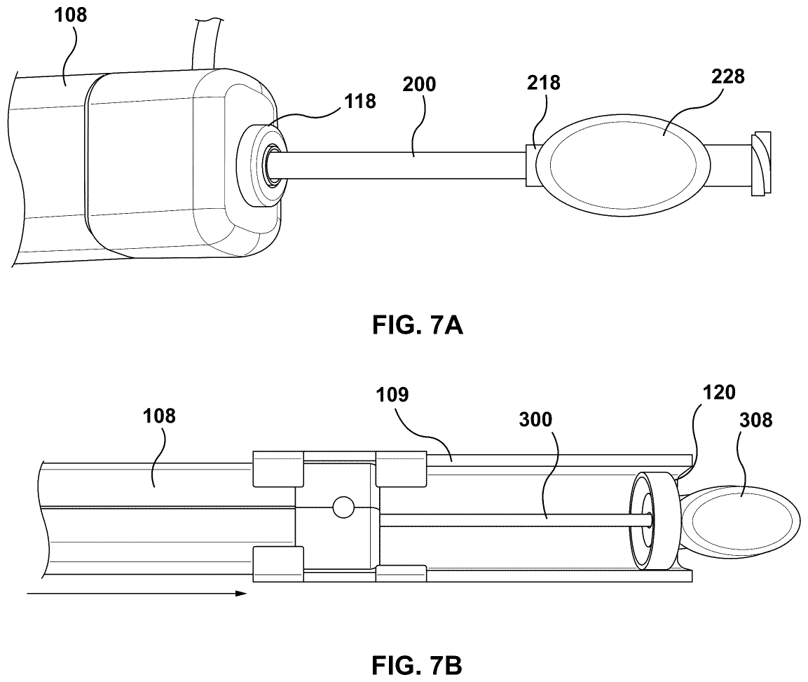

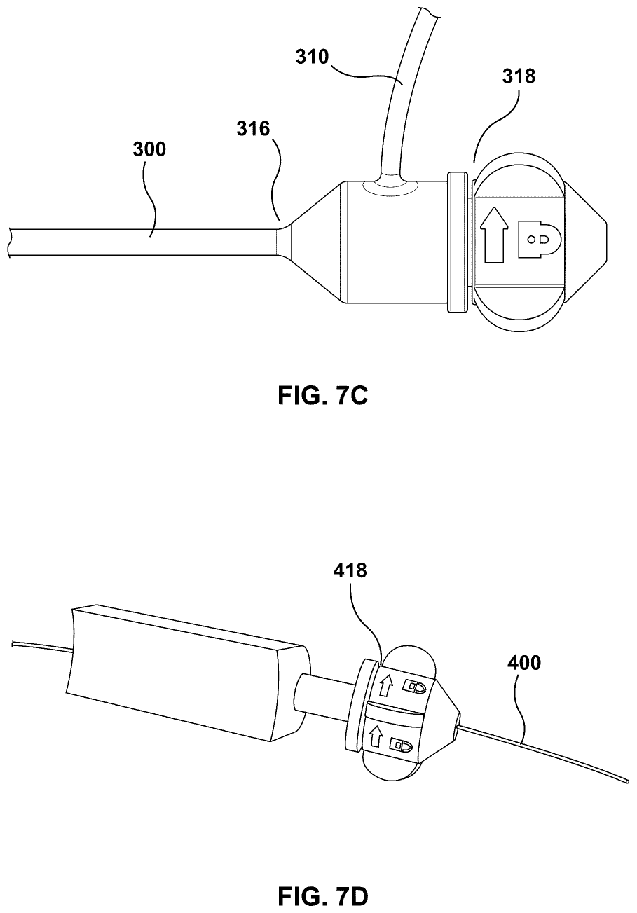

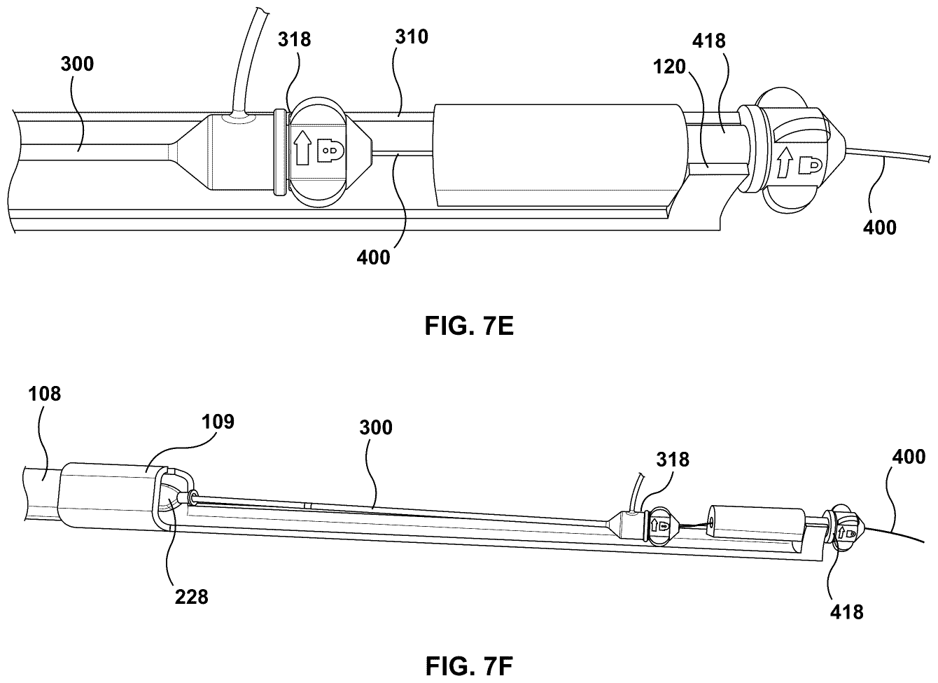

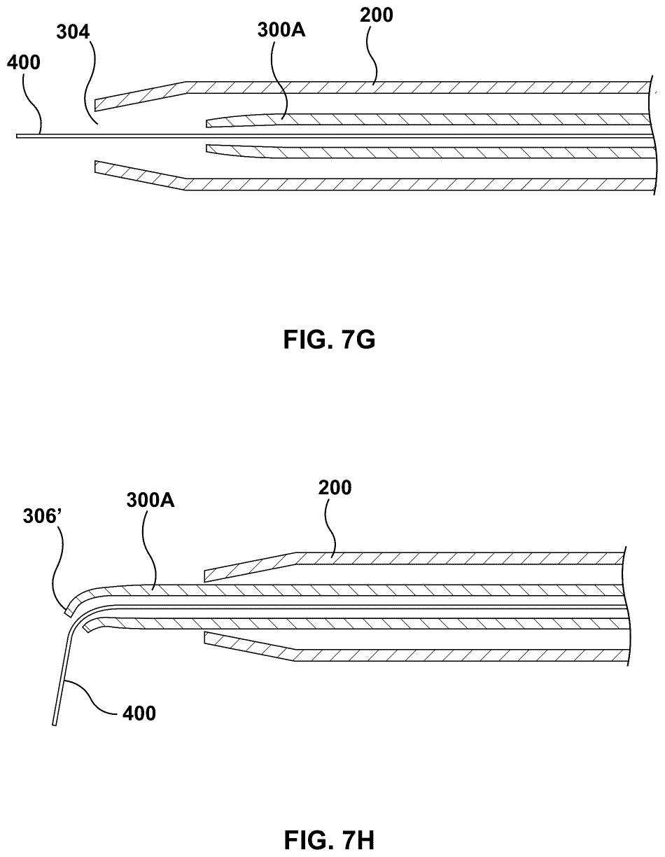

In some embodiments, dilator 200 comprises a hemostatic valve (not shown) on the proximal end 216. In some embodiments, dilator 200 comprises a hub 228 connected to the dilator proximal end 216, shown in FIG. 7A. In one embodiment, hub 228 comprises a hemostatic valve (not shown) for creating a seal between dilator 200 and another device inserted into dilator 200, for example catheter 300 or puncture wire 400. In some embodiments, hub 228 can connect to a valve 124, for connecting to additional external devices. In some embodiments, hub 228 provides a grip for an operator and may comprise locking features and/or alignment features described herein. Any of dilator 200A, 200B, 200C, or 200D may comprise a hemostatic valve or hub 228 described herein.

### Alignment Features

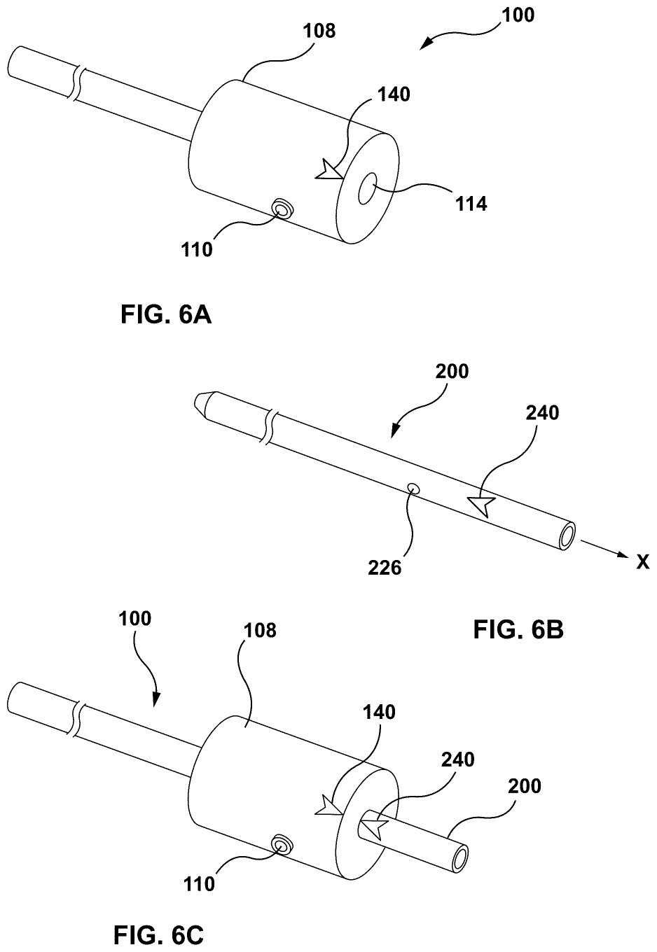

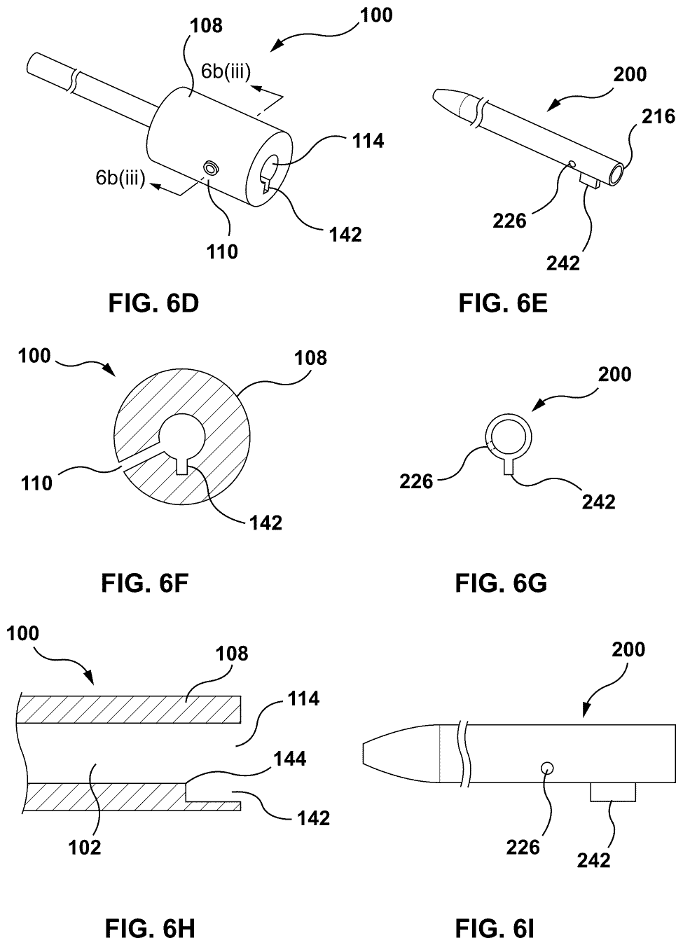

With reference to FIGS. 6A to 6C, in one embodiment of the present invention, one or both of sheath 100 or dilator 200 may include one or more alignment features on or near sheath proximal end 116 and/or dilator proximal end 216 respectively. Alignment features may be used to ensure one or more devices of the assembly 1000 is in an aligned configuration. In one embodiment, sheath handle 108 comprises alignment feature 140 and dilator 200 comprises alignment feature 240. Alignment features 140 and 240 may substantially comprise markings that indicate the required placement of the dilator 200, relative to sheath 100 in a longitudinal direction, i.e., along a longitudinal axis X, and the required rotation of dilator 200 relative to sheath 100, i.e., about a longitudinal axis X. In other words, when alignment features 140 and 240 are brought together, as shown in FIG. 6C, an aligned configuration is achieved, and side port 110 is aligned with secondary proximal opening 226 longitudinally and rotationally. In another embodiment, alignment feature 240 is located on a dilator hub (described herein).

In some embodiments, alignment features 140 and 240 may comprise markings to indicate i) a longitudinal alignment, ii) a rotational alignment, or iii) a longitudinal and rotational alignment.

In another embodiment, alignment features 140 and/or 240 may substantially comprise markings that indicate a longitudinal direction alignment only, because rotational alignment about a longitudinal axis is not required, for example, with dilator 200C described herein. An aligned configuration is achieved when reduced diameter portion 250 is aligned in a longitudinal direction with side port 110.

In another embodiment, shown in FIGS. 6D to 6I, alignment features may comprise one or more guiding features. In one embodiment, guiding features comprise one or more grooves, slots, or protrusions such that dilator 200 is always rotationally aligned when inserted into sheath 100. In one example, sheath 100 comprises groove 142 associated with the proximal opening 114. Dilator 200 comprises protrusion 242 at or near the proximal end 216. Protrusion 242 is configured to be insertable into groove 142 such that a rotational alignment is achieved. FIG. 6F shows a cross-sectional view of guiding features on sheath 100, at the location of the side port 110. FIG. 6G shows a rear view of dilator 200 (secondary proximal opening 226 shown in dashed lines).

In some embodiments, shown in cross-section views in FIG. 6H, one or more guiding features may comprise a stop feature such that movement of dilator 200 stops at a longitudinally aligned position. For example, sheath 100 comprises stop feature 144, configured such that dilator 200 is insertable into sheath primary lumen 102 until protrusion 242, shown in FIG. 6I abuts stop feature 144, and dilator 200 cannot be advanced any further. When protrusion 242 abuts stop feature 144, a longitudinal alignment is achieved. In some embodiments, alignment features provide an operator with tactile or audible feedback when an aligned position is achieved, for example, a “click”, such as for a snap-fit connection.

### Locking Features

Referring now to FIG. 7A, in one embodiment, sheath 100 and/or dilator 200 include one or more locking features on sheath proximal end 116 and/or dilator proximal end 216. In some embodiments, locking features may be located on sheath handle 108 and/or dilator hub 228. Locking features allow for the assembly 1000 to be alternated between a “locked state” and an “unlocked state”. In a locked state, longitudinal movement between two devices is limited, and in an unlocked state, the two devices may move longitudinally with respect to each other. In some embodiments rotational movement is limited in a locked state. For clarity, “limited” may mean limited entirely, i.e., movement is prohibited or prevented, or “limited” may mean substantially inhibited without almost zero movement. For additional clarification, the terms “locked” and “secured” may be used interchangeably.

In one example, sheath handle 108 comprises locking feature 118 and hub 228 comprises locking feature 218. In one specific example, locking features 118 and 218 are configured to form a snap-fit engagement with each other, whereby the dimensions and materials of locking features 118 and 218 snap together such that they do not to come apart without manual manipulation. When snapped together, sheath 100 and dilator 200 are in a locked state and one will not move in a longitudinal direction (distally or proximally) relative to the other. Conversely, in an unlocked state, dilator 200 can move within sheath primary lumen 102 in a longitudinal direction. In some embodiments, dilator 200 can rotate with respect to sheath 100 in a locked state. In another embodiment, rotational movement of dilator 200 in a locked state is limited.

In other embodiments, locking features 118/218 may be any other devices or mechanisms known in the art such that they prevent movement of an inner device relative to an outer device. In one example, locking features 118/218 comprise a clip like locking mechanism. In another example, the locking features 118/218 comprise a rotational or twist-lock locking mechanism where locking/unlocking is done by rotating one of the sheath 100 or dilator 200 into a locked/unlocked state. In some examples, one or more locking features is a Touhy-Borst luer lock. In another example, locking features 118/218 comprise a push mechanism.

In a locked state, an operator may be able to manipulate the sheath 100 and dilator 200 simultaneously, using one hand, e.g., advance both devices distally or retract both devices proximally.

In some embodiments, the locked state corresponds to an aligned configuration, meaning the flush port 110 and secondary proximal opening 226 are fluidically connected. In other embodiments, a locked state corresponds to a longitudinal alignment, but not necessarily a rotational alignment. In other embodiments, a locked state corresponds to a longitudinal alignment and a rotational alignment.

In one embodiment, alignment features 140/240 and/or locking features 118/218 may be such that a locked state always corresponds to aligned configuration. In other words, the only way to achieve an aligned configuration is to put the devices in a locked state. In such an embodiment, locking two devices comprises aligning the devices, such that a locked state always results in two devices being in an aligned configuration. In other words, in order to lock the devices together, they must be aligned. For example, as shown in FIGS. 6D to 6I, locking features 118/218 are configured to engage only after protrusion 242 is inserted into groove 142 and protrusion 242 abuts stopping feature 144.

For clarification, in some embodiments, two devices may be i) not aligned and not locked, ii) aligned but not locked, iii) locked but not aligned, or iv) locked and aligned. In one embodiment, where a locked state always corresponds to an aligned configuration, the devices may alternate between two states: i) unlocked and non-aligned and ii) locked and aligned.

Referring now to FIG. 7B, in another embodiment, sheath 100 may comprise a second/secondary locking feature for securing longitudinal movement of a third device, for example catheter 300. In one example, sheath comprises a handle extender 109, configured to attach to the proximal end of handle 108 and catheter 300 comprises a catheter handle 308 coupled to the catheter proximal end 316. Handle extender 109 comprises secondary locking feature 120, which is configured to interact with a portion of catheter handle 308. In another embodiment, secondary locking feature 120 is defined by sheath handle 108.

Secondary locking feature 120 is configured to receive a portion of catheter handle 308, such that longitudinal movement of catheter 300, relative to sheath 100, is limited. In other words, sheath 100 and catheter 300 are locked together. In some embodiments, rotational movement of catheter 300, with respect to sheath 100, is limited while in a locked state.

In one example, sheath 100, dilator 200, and catheter 300 are locked together and there is no longitudinal movement of any device with respect to any other device. In some embodiments, rotational movement of all three devices, with respect to each other, is limited while in a locked state. In other embodiments, rotational movement of only one device is limited.

In another embodiment, the assembly 1000 comprises a locking feature for securing catheter 300 to puncture wire 400, which may be used in conjunction with the locking features described above. In one example, shown in FIG. 7C, catheter 300 comprises a locking feature 318 on a proximal end 316 to limit movement of the puncture wire 400. Locking feature 318 may be any device known in the art, for example a push to lock/unlock device or twist to lock device.

In one example, the locking feature 318 is a Tuohy-Borst connector. In some embodiments longitudinal movement is limited. In other embodiments, longitudinal and rotational movement of puncture wire 400 is limited with respect to catheter 300. In some embodiments, locking feature 318 is configured to be received within sheath secondary locking feature 120, as described herein for catheter handle 308.

In some examples, all four devices, sheath 100, dilator 200, catheter 300, and puncture wire 400 may be locked together, such that no device is able to move longitudinally with respect to any other device. In other embodiments, rotational movement of all devices with respect to each other is limited. In other embodiments, rotational movement of one or two devices is limited. In one example, sheath locking feature 118 limits movement of dilator 200 with respect to sheath 100, sheath locking feature 120 limits movement of catheter 300 with respect to sheath 100, and catheter locking feature 318 limits movement of puncture wire 400 with respect to catheter 300, thereby securing all four devices together.

In other examples, sheath 100 and dilator 200 may be locked together, and catheter 300 and puncture wire 400 may be locked together. This may be done at specific steps during a TIPS procedure, for example during the puncture where a tract is created through the liver. (In some examples, RF energy is used to create the tract. In other examples, mechanical energy is used to create the tract.) An operator may hold the locked sheath-dilator in one hand and the locked catheter-puncture wire in the other.

In another embodiment, shown in FIG. 7D, puncture wire 400 comprises a locking feature 418. In one embodiment, locking feature 418 comprises a twist-lock mechanism and may be configured to be received within sheath secondary locking feature 120. While received within sheath secondary locking feature 120, locking feature 418 may be operable to allow or limit movement of puncture wire 400 with respect to locking feature 418.

FIG. 7E shows puncture wire 400 comprising locking feature 418 received within sheath secondary locking feature 120 on sheath handle extender 109. Puncture wire 400 is received within catheter 300, passing through catheter locking feature 318. In such an embodiment, puncture wire locking feature 418 can be secured to puncture wire 400, for example via a twist-lock mechanism. puncture wire locking feature 418 is further configured to be secured within sheath secondary locking feature 120, such that movement of puncture wire locking feature 418 is limited in a longitudinal direction, and further, when puncture wire locking feature 418 is secured to puncture wire 400, longitudinal movement of puncture wire 400 is limited with respect to sheath 100/sheath handle 108.

Any or all locking features may be used in a method, described herein, to advance the catheter 300 and puncture wire 400 together during the TIPS procedure, for example during the puncture step of a TIPS procedure, and then to also advance the catheter with respect to the wire after the puncture step. In other words, catheter 300 and puncture wire 400 are locked or secured together during the puncture, then unlocked so that the catheter 300 may advance over puncture wire 400.

FIG. 7F shows portions of all four devices of telescoping assembly 1000, including locking features of each.

In some embodiments, catheter 300 is an angled catheter 300A and has a flexible curve at the distal end 306′. FIGS. 7G and 7H show cross-sectional views of the puncture wire 400 received within angled catheter 300A, which is received within dilator 200. The flexibility of the distal end 306′ is such that when angled catheter 300 is retracted into dilator 200 (FIG. 7G), to a first position, angled catheter distal end 306′ and puncture wire 400 exit the dilator distal opening 204 in a substantially straight or horizontal configuration. In other words, puncture wire 400 extends along a longitudinal axis of the dilator 200.

When angled catheter 300A is extended out of dilator distal opening 204, to a second position, angled catheter 300A adopts a curved configuration, shown in FIG. 7H. puncture wire 400 distal end also adopts a curved configuration. The material properties of the dilator 200 and/or angled catheter 300 are configured to provide the curved configuration and the substantially straight configuration, depending on where the angled catheter 300A is received within the dilator 200.

In some embodiments, catheter 300/300A and/or puncture wire 400 comprise alignment features, described herein, on the proximal ends (not shown) to indicate where the devices need to be relative to each other (longitudinally) such that they are in the first position and/or the second position.

### Elongate Aperture

At times during a TIPS procedure, it may be advantageous to lock two devices together and also allow for controlled movement of a third device. For example, puncture wire 400 is within catheter 300, and catheter 300 is within dilator 200. It may be desirable to lock puncture wire 400 to dilator 200 and have the ability to move catheter 300 relative to the locked puncture wire-dilator.

One example during a TIPS procedure where this may be desirable is after the puncture wire 400 has created a tract through the liver 10 but has not yet fully tracked into the portal vein 16. In one embodiment, angled catheter 300A is used to angle the puncture wire 400 after the puncture, so that puncture wire 400 can track into the portal vein 16.

After a tract has been created through the liver 10, other devices need to be advanced into the portal vein 16. In order to advance other devices, a guidewire needs to sufficiently track into portal vein 16. Puncture wire 400 may act as a guidewire in some embodiments, but in some cases, it is difficult for the puncture wire 400 to track into the portal vein 16 because of its stiffness properties. Puncture wire 400 requires a certain stiffness to puncture through the liver 10. Once puncture wire 400 enters portal vein 16, it may run into the portal vein wall and not track, i.e., it will not turn or curve into the portal vein 16 far enough to continue the procedure.

One way to overcome this problem is to use an angled catheter 300A. Angled catheter 300A may be flexible enough that when retracted, puncture wire 400 is straight enough for the puncture, but when catheter 300A advances over puncture wire 400, puncture wire 400 is able to curve and track into the portal vein 16.

In order to advance the catheter 300A so that puncture wire 400 will track into the portal vein 16, an operator needs to hold the puncture wire 400, hold the sheath 100 and/or dilator 200, and advance (or push) the catheter 300A. From the perspective of a single operator, one hand is used to hold the dilator 200 (or sheath-dilator combination), and the other hand is used to hold the puncture wire 400. There is no hand free to advance the catheter 300A. If the puncture wire 400 and dilator 200 are locked together, the operator could use one hand to hold both the puncture wire 400 and dilator 200 and have the other hand free to advance the catheter 300A.

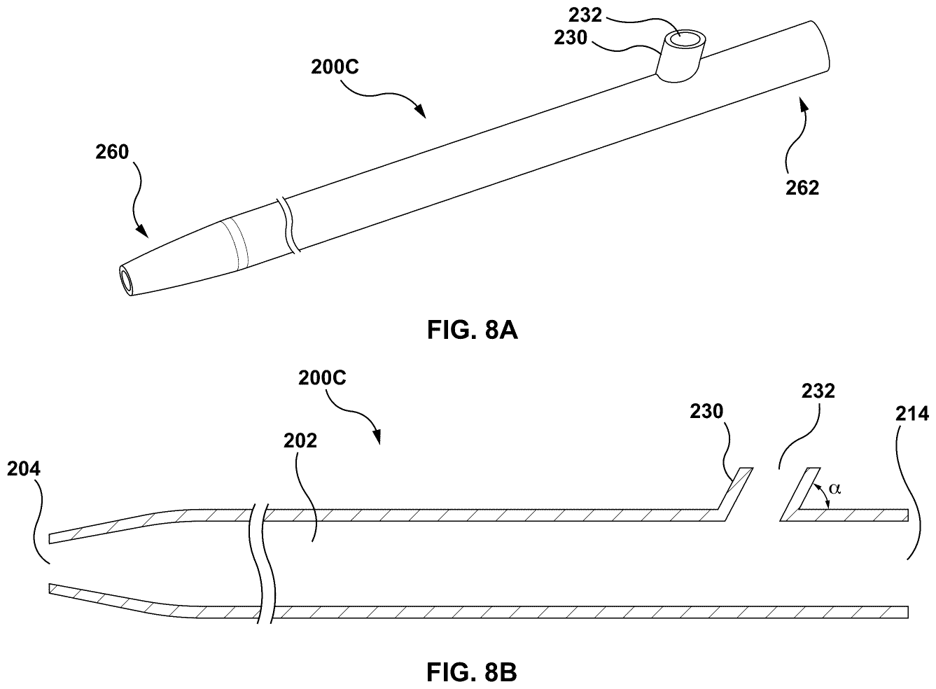

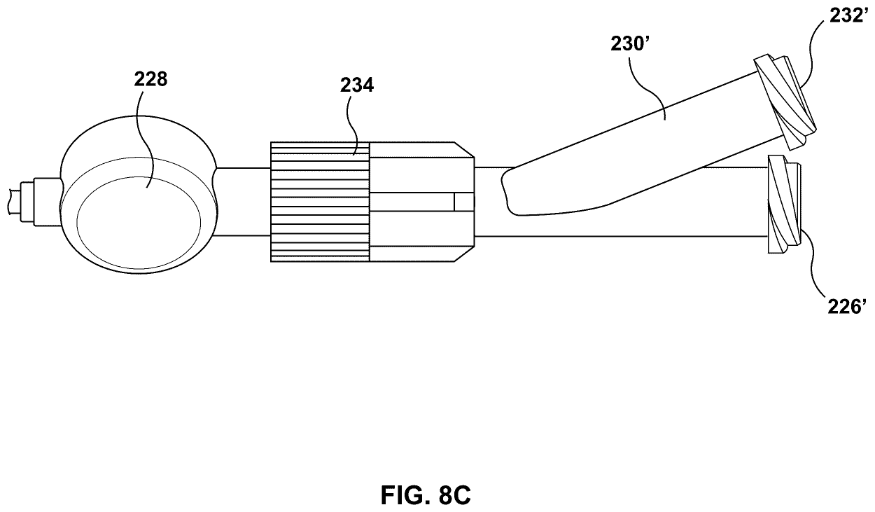

Referring now to FIGS. 8A and 8B, in one embodiment, dilator 200C defines primary lumen 202 extending between distal opening 204 in a distal region 260 and a proximal opening 214 in a proximal region 260. Dilator 200C further defines a side port 230, located in the proximal region 260, in fluid communication with primary lumen 202. In one embodiment, side port 230 defines a short passageway set on an angle relative to the longitudinal axis of primary 202, as shown as angle a. Side port 230 further defines a side opening 232, that is large enough to accommodate another device, for example puncture wire 400. Dilator 200c may also comprise a hemostatic valve (not shown) at side opening 232.

In another embodiment, shown in FIG. 8C, dilator 200 comprises hub 228 coupled to proximal end 216. Hub 228 is connected to multi-channel attachment, such as Y-connector 234. Y-connector 234 is in fluid communication with dilator primary lumen 202 and defines a proximal opening 226′ for receiving a second device, such as catheter 300, and side port 230′ defining side opening 232′ for receiving a third device, such as puncture wire 400.

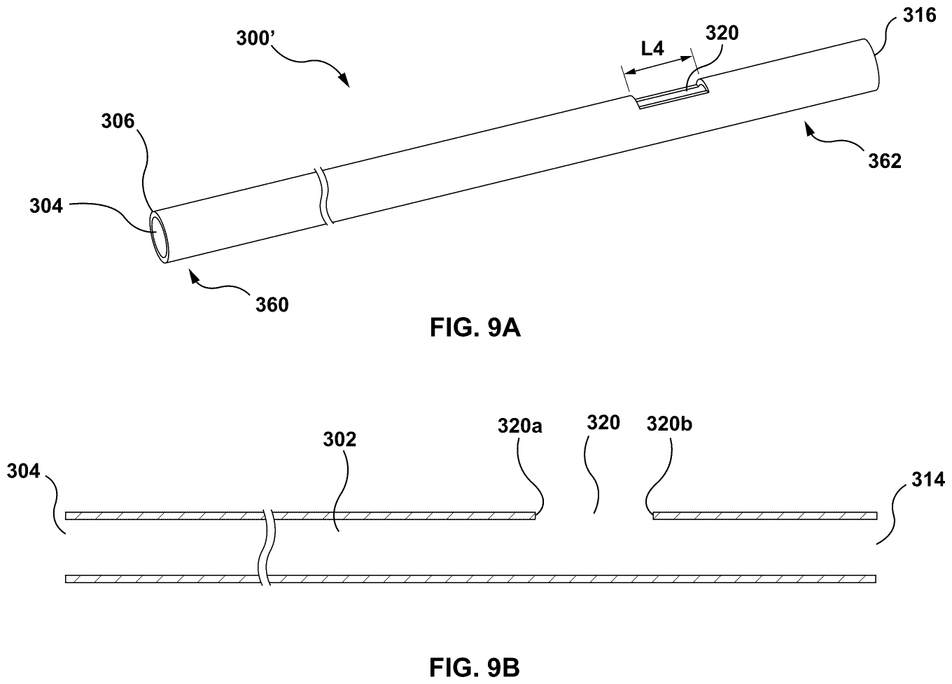

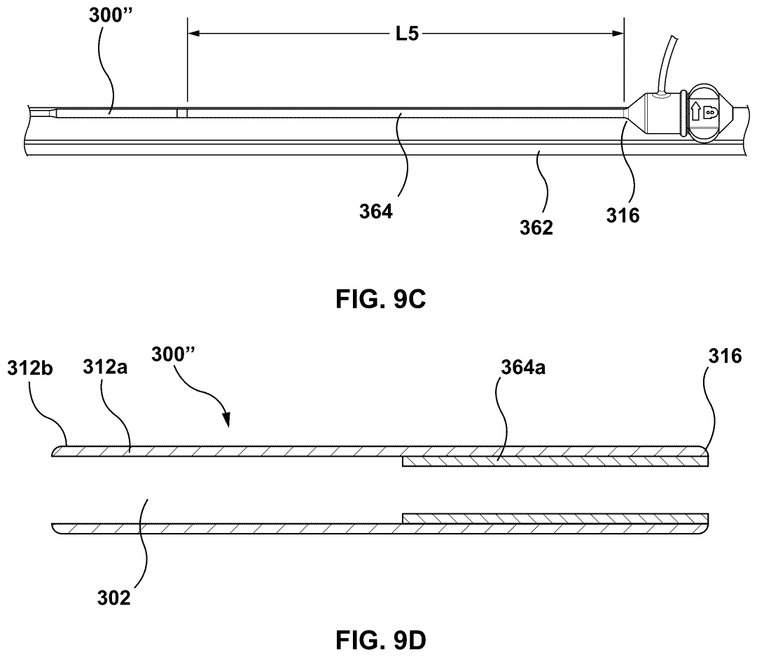

Referring to FIGS. 9A to 9B, in one embodiment catheter 300′ comprises a distal region 360 and a proximal region 362. Catheter 300′ further defines primary lumen 302 extending between distal opening 304 at a distal end 306 and proximal opening 314 at proximal end 316. Primary lumen 302 is large enough to receive an elongate device, such as puncture wire 400. Catheter 300′ further comprises a sidewall in the proximal region 362 and further defines an elongated aperture or elongate opening 320. Elongate opening 320 spans a distance L4 and is in communication with lumen 302. In one specific example L4 may be within a range of 4 to 6 cm. Elongate opening 320 extends from elongate opening distal end 320a to elongate opening proximal end 320b. Elongate opening 320 is sized to receive a third elongate device, such as a puncture wire 400, such that puncture wire 400 extends from elongate opening 320, through primary lumen 302, to distal opening 304. In other words, puncture wire 400 enters at elongate opening 320 and exits at distal opening 304.

In another embodiment shown in FIG. 9C, catheter 300″ comprises a stiff proximal segment 364 located in proximal region 362. Stiff proximal segment 364 extends from proximal end 316 (distally) to a distance L5. In one example, distance L5 is between 18 cm and 22 cm. In another example, distance L5 is between 25 cm and 29 cm. Stiff proximal segment 364 is configured such that it is relatively inflexible and remains rigid, so that it may be easily manipulated by an operator. Catheter 300″ may be configured such that when stiff proximal segment 364 extends out of the sheath proximal opening 114 or dilator 200 proximal opening 214, it remains rigid. In other words, it does not droop or curve, but remains substantially straight, along a longitudinal axis of the assembly 1000. The stiff proximal segment 364 may also act to passively “lock” the catheter 300″ to the dilator 200. In other words, if the catheter proximal end is floppy, letting go of the catheter may result in the catheter sliding out of the dilator. However, with a rigid proximal end, an operator may be able to let go of catheter 300″ and it will remain within the dilator 200, in other words, without moving longitudinally.

In one embodiment, catheter 300″ comprises a rigid component 364a to provide stiffness in the stiff proximal segment 364, for example a hypotube made of steel or metal. The hypotube may be located within an inner wall 312a of catheter 300″, shown in a cross-section view in FIG. 9D, in other words, located within catheter 300″ primary lumen 302. In other embodiments, rigid component 364a is located within the catheter 300″ walls, in other words between inner wall 312a and outer wall 312b. In another embodiment, rigid component 364a is located on the outside of catheter 300″, in other words, located over the outer wall 312b (not shown). In one embodiment, rigid component 364a is a stainless-steel tube. In another embodiment, rigid component 364a is a laser-cut hypotube. In another embodiment, stiff proximal segment 364 comprises a braided region inside a high modulus polymer region. In one example, the polymer is part of the Nylon 12 family, such as Grilamid® or Vestamid®. approximately 63D-55D or 63D-63D.

In other embodiments, catheter 300″ comprises one or more gripping features in the stiff proximal segment 364 (not shown) to allow an operator to grip the proximal region of catheter 300″ so that it can be advanced or retracted.

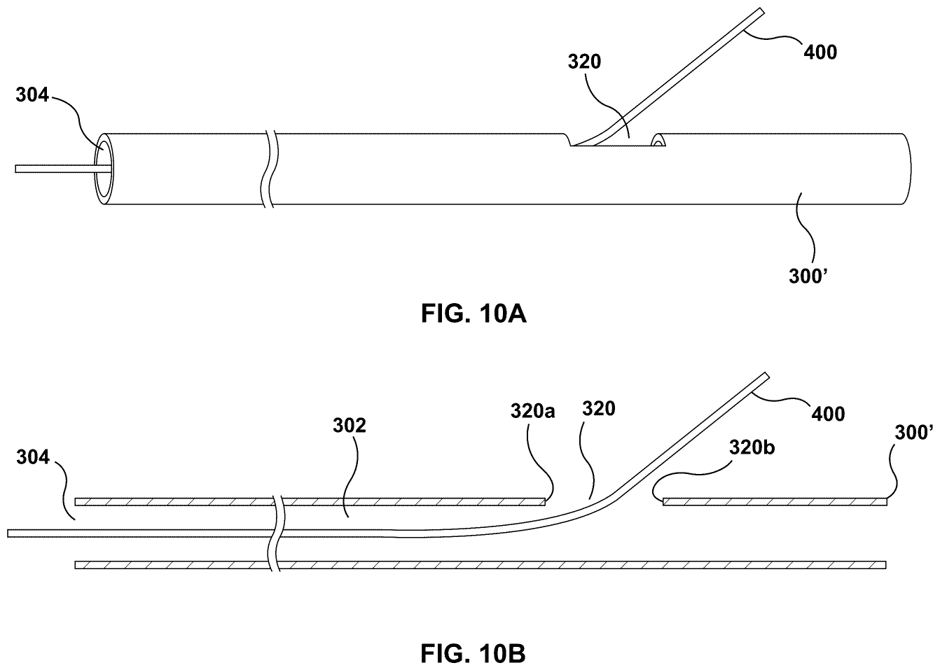

Referring to FIGS. 10A and 10B, puncture wire 400 is shown entering the elongate opening 320 of catheter 300′. Puncture wire 400 is configured to be insertable into primary lumen 302 via elongate opening 320. Puncture wire 400 is further configured to be moveable in a longitudinal direction within lumen 302.

When catheter 300′ is inserted in dilator primary lumen 202, side port 230 can be aligned with catheter elongate opening 320 such that a device, for example puncture wire 400, can be inserted into side opening 232, pass through elongate opening 320, enter catheter primary lumen 302 and exit through catheter distal opening 304.

In a first position, puncture wire 400 abuts elongate opening distal end 320a. Catheter 300′ may be advanced distally with respect to puncture wire 400 until puncture wire 400 reaches a second position, where puncture wire 400 substantially abuts elongate opening proximal end 320b.

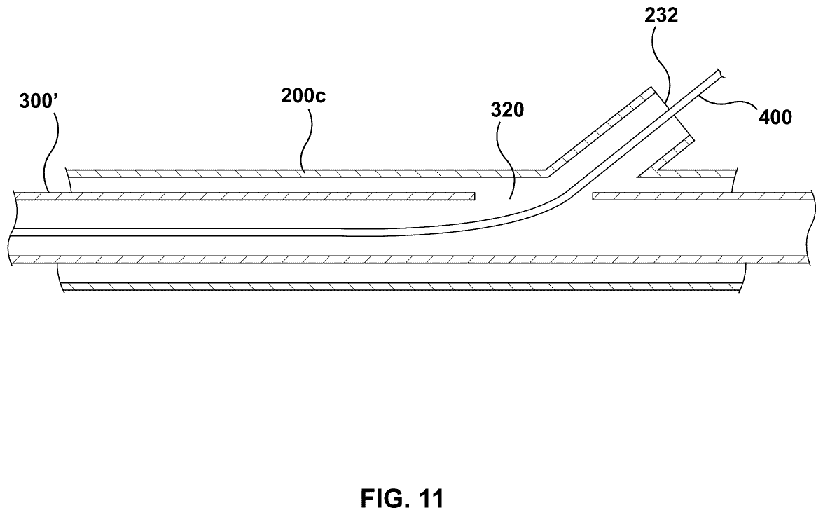

FIG. 11 shows a cross-section view of puncture wire 400 received within catheter primary lumen 302, which is received within dilator primary lumen 202. Puncture wire 400 can be inserted through dilator side opening 232, then through catheter elongate opening 320, and exit out of catheter distal opening 304. Catheter 300′ extends between dilator 200c distal opening 204 and dilator proximal opening 214.

In one embodiment, dilator side port 230 may comprise one or more locking features, described herein, that may prevent movement of puncture wire 400 with respect to dilator 200C. While in this locked state, catheter 300 may be advanced/retracted distally/proximally with respect to the locked dilator-puncture wire. The distance catheter 300′ can be advanced or retracted is restricted to the dimensions of the elongate opening 320, for example, limited to L4. In other words, longitudinal movement of the catheter 300′ is limited to the length of elongate opening 320. In another embodiment, an external locking feature is connected to dilator side port 230 for securing the puncture wire 400, for example, a Tuohy-Borst connector or other locking feature similar to locking feature 318.

In some embodiments, dilator 200C may be received with sheath 100 forming a telescoped assembly 1000′ (not shown), and further, sheath 100 may comprise one or more locking features, described herein, for restraining movement of the dilator and/or other devices.

In another embodiment, sheath 100 may comprise a sheath side port (not shown) similar to dilator side port 230 described herein.

### Advancement Mechanisms

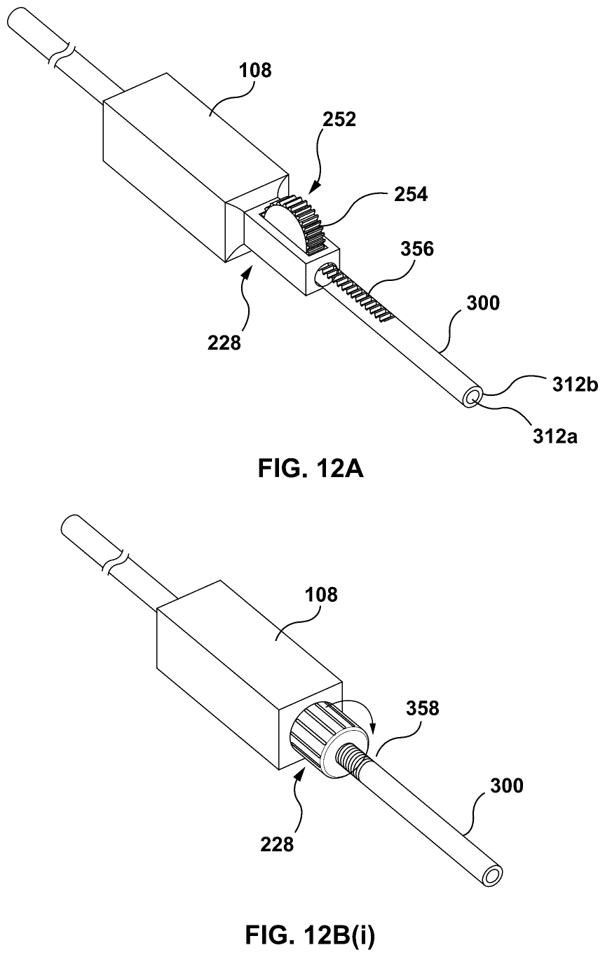

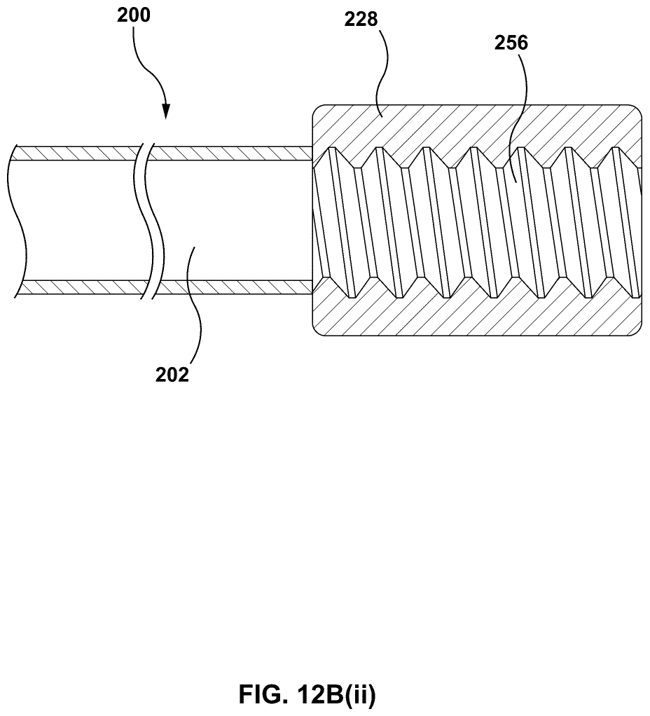

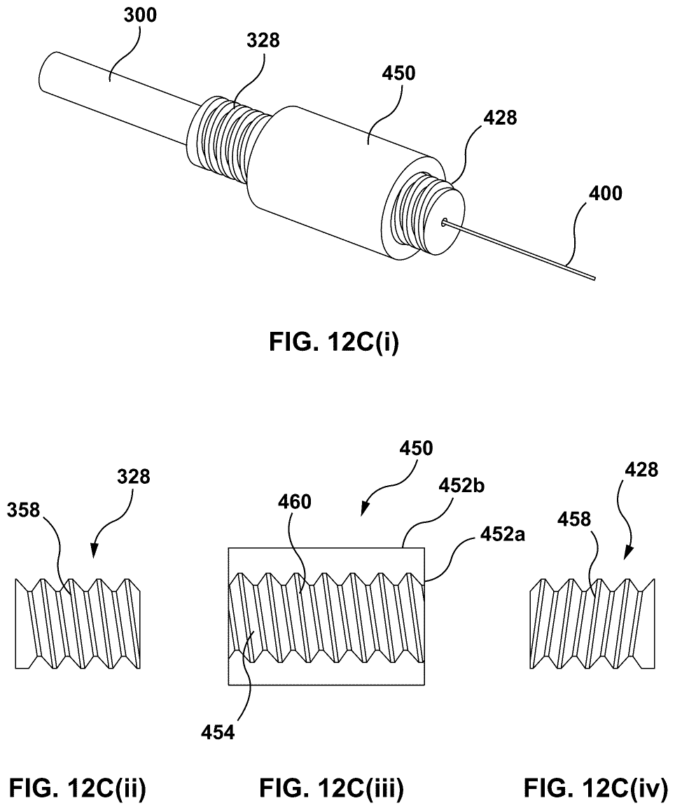

Referring to FIG. 12A, in another embodiment, the assembly 1000 comprises one or more advancement mechanisms for advancing one of the devices. In one embodiment, dilator hub 228 comprises advancement mechanism 252, which comprises gear 254. Gear 254 aligns with and fits into grooves or teeth 356, defined by the catheter outer wall 312b, as shown in a cross-section view in FIG. 12A(iii). In use, an operator may grip the sheath handle 108 with one hand and use the thumb of the same hand to operate advancement mechanism 252 to advance the catheter 300. In such an embodiment, rotation of gear 254 moves catheter 300 in a longitudinal direction. This allows the operator's second hand free to hold the puncture wire 400. In such an embodiment, an operator can manipulate three devices with one hand and a fourth device with the other.

In another embodiment, assembly 1000 comprises an advancement mechanism that is configured so that rotational movement about a longitudinal axis causes longitudinal movement of one or more devices.

Referring to FIG. 12B, in another embodiment, catheter outer wall 312b defines an outer threaded region 358. As shown in a cross-section view in FIG. 12B(ii), dilator hub 228 defines an inner threaded region 256. Outer threaded region 358 of the catheter 300 is configured to be received within inner threaded region 256 of dilator hub 228, such that rotation of the dilator hub 228 translates into longitudinal movement of catheter 300.

In such an embodiment, the dilator hub 228 can be longitudinally locked into sheath handle 108, and an operator can hold sheath handle 108 with one hand, and rotate dilator hub 228 using the thumb of the same hand, thereby advancing the catheter 300. The operator's second hand can hold the puncture wire 400, which is received within catheter 300.

Referring to FIG. 12C(i) to 12C(iv), in another embodiment, a rotational mechanism is provided for advancing the catheter 300 with respect to puncture wire 400. In such an embodiment, catheter 300 comprises threaded hub 328 coupled to the catheter proximal end 316. Threaded hub 328 defines an outer threaded region 358 on the outer surface. In another example, catheter 300 outer wall 312b defines the outer threaded region 358.

Puncture wire 400 is coupled to a puncture wire threaded hub 428. Threaded hub 428 defines an external threaded region 458 on its outer surface and comprises a locking feature, described herein, for securing threaded hub 428 to puncture wire 400, for example a twist-lock mechanism.

Rotational mechanism 450 comprises an inner wall 452a and outer wall 452b, which define an internal lumen 454 and an internal threaded region 460. In one embodiment, rotational mechanism 450 defines a cylinder shape, and may include a gripping feature (not shown). Internal lumen 454 is configured to receive threaded hubs 328 and 428. In one example, rotational mechanism 450 is partially secured to catheter threaded hub 328 and puncture wire threaded hub 428.

In one embodiment, internal threaded region 460 is configured to engage with outer threaded region 358 and external threaded region 458, such that rotation of rotational mechanism 450 in a first direction causes catheter 300 to move distally and causes puncture wire 400 to move proximally relative to the device handle, and rotation of rotational mechanism 450 in a second direction causes catheter 300 to move proximally and causes puncture wire 400 to move distally relative to the device handle.

An operator may then hold the locked sheath 100 and dilator 200 with one hand and hold the rotational mechanism 450 with the other, thereby having the ability to manipulate the catheter 300 and puncture wire 400. The operator can twist or rotate the rotation mechanism 450 about a longitudinal axis to advance the catheter 300 relative to puncture wire 400. The operator can also advance rotational mechanism 450 distally while twisting it to ensure puncture wire 400 remains in substantially the same place.

### Method for Manipulating Fluids During a Tips Procedure

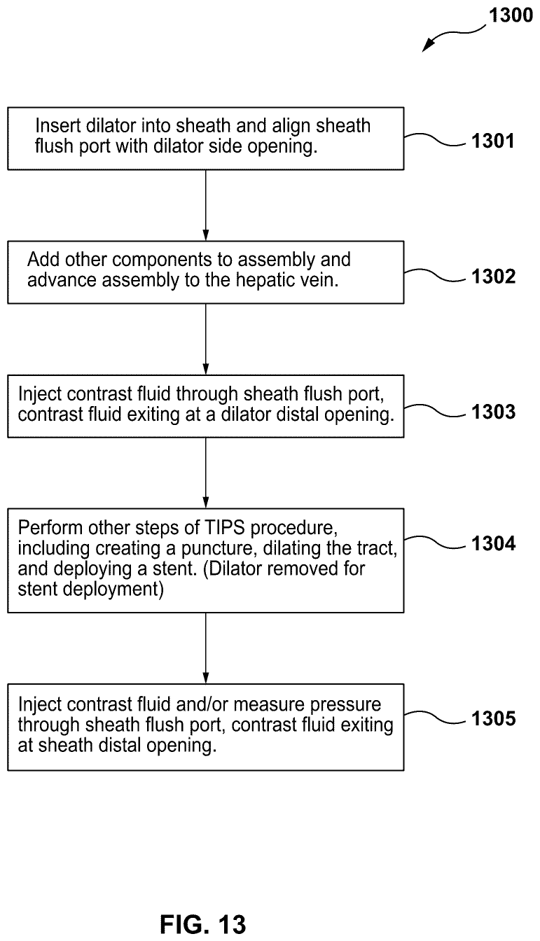

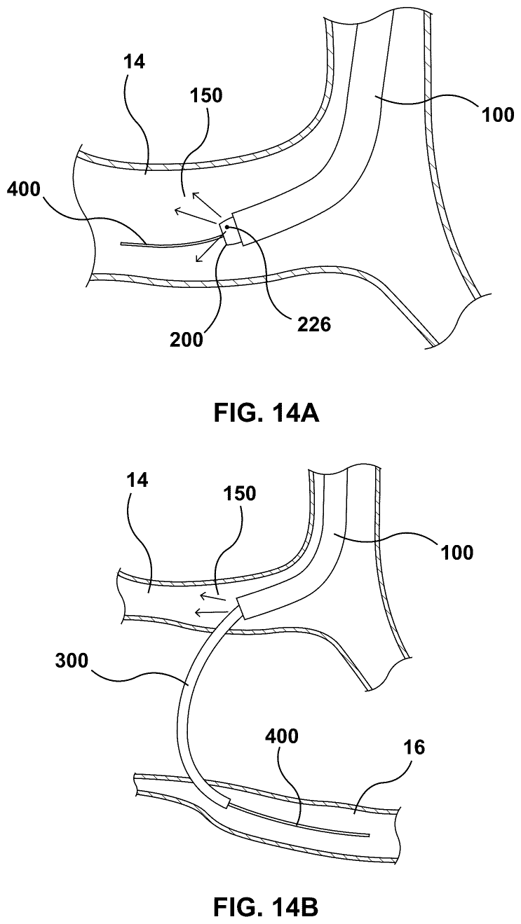

FIG. 13 shows a method for interacting with fluids in blood vessels using a telescoping assembly during a TIPS procedure. Interacting with fluids may include, but is not limited to, injecting contrast, aspirating, or measuring a pressure within a blood vessel. Each of which may be performed multiple times during a TIPS procedure. The method may be better understood by referring also to FIGS. 14A and 14B, which illustrate the devices in use during the procedure.

Method 1300 begins at step 1301, dilator 200 is inserted into sheath primary lumen 102 and put into an aligned configuration, i.e., sheath side port and dilator side opening are aligned. Sheath 100 and dilator 200 may optionally be locked together as described herein.

At step 1302, catheter 300 and/or puncture wire 400 are inserted into the dilator lumen 202, and the assembly 1000 may be advanced through the inferior vena cava towards the hepatic vein 14.

At step 1303, with reference to FIG. 14A, in order to confirm the position of the distal end of the assembly 1000, contrast fluid is injected into the hepatic vein 14 and surrounding blood vessels using an injection device fluidically connected to sheath flush port. The distal end of assembly 1000 can be visualized using known imaging modalities. The contrast fluid is injected through the flush port, travels through a lumen in the dilator and exits at a distal opening of the dilator, for example secondary lumen 222 and secondary distal opening 224. Contrast fluid is represented by arrows 150 in FIG. 14A.

At step 1304, one or more additional steps of a TIPS procedure may be performed, such as puncturing the liver with the puncture wire 400 and creating a tract through the liver with dilator 200, and then delivering a stent (not shown). When the stent is placed across the tract, the dilator 200 is removed from the sheath 100/assembly 1000.

At step 1305, with reference to FIG. 14B, dilator 200 has been removed from the assembly 1000 and catheter 300 and puncture wire 400 are in the portal vein 16, extending through a tract in the liver. The distal end of dilator 200 remains in the hepatic vein 14. The hepatic vein 14, tract and/or surrounding blood vessels may need to be visualized again, which requires injecting additional contrast fluid. Using the same injection device connected to sheath flush port as step 1103, contrast fluid is injected. The contrast fluid travels through sheath flush port, through sheath primary lumen 102, and exits at the sheath distal opening 104. Contrast fluid is represented by arrows 150 in FIG. 14B.

In addition to injecting contrast, pressure in the hepatic vein 14 could also be measured using the sheath primary lumen and the sheath flush port. Contrast fluid could be injected into the portal vein 16, and pressure could be measured in the portal vein 16, using catheter 300.

Method 1300 allows an operator to use a single fluid manipulation device coupled to the sheath 100, to manipulate fluids regardless of whether the dilator 200 is received within sheath 100.

### Method for Advancing Devices During a Tips Procedure

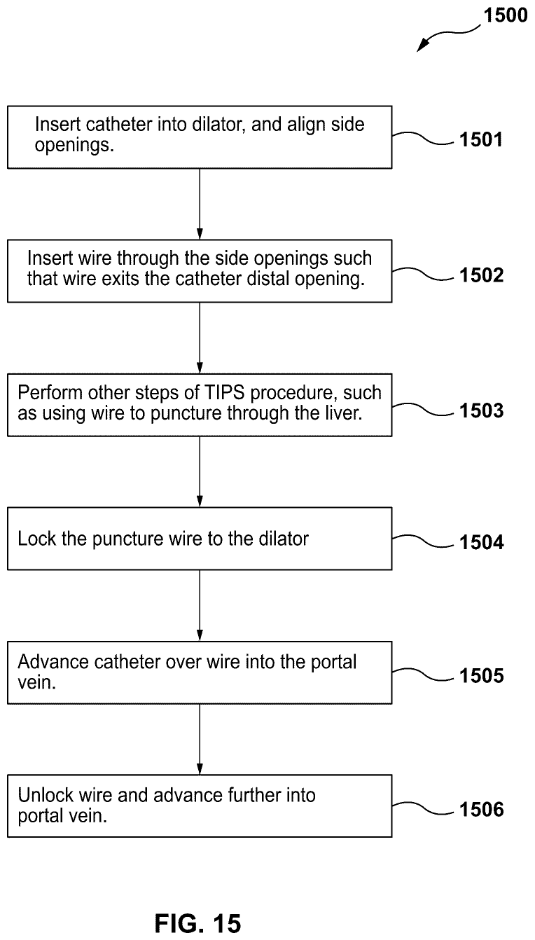

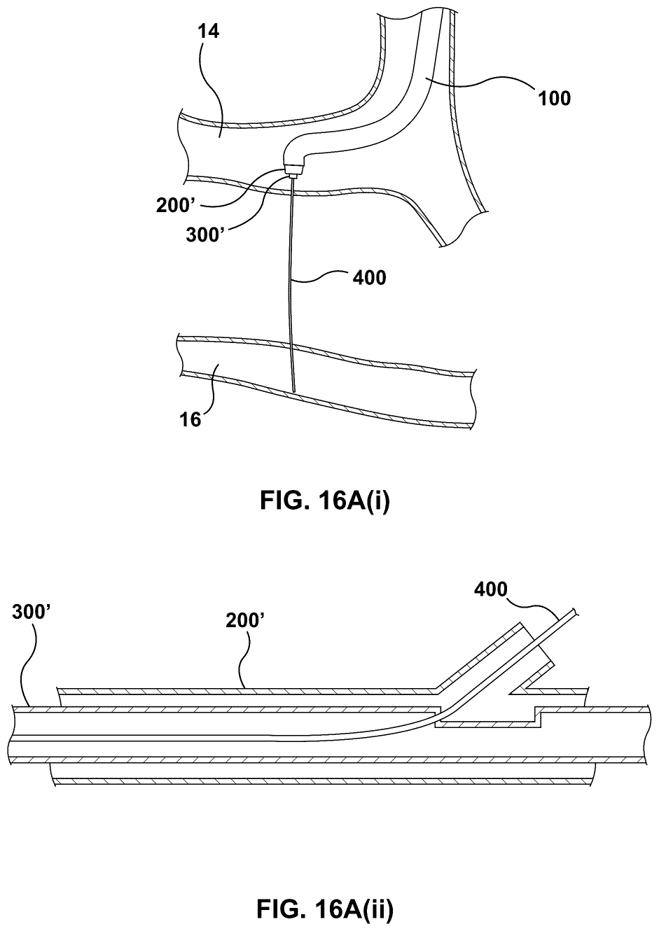

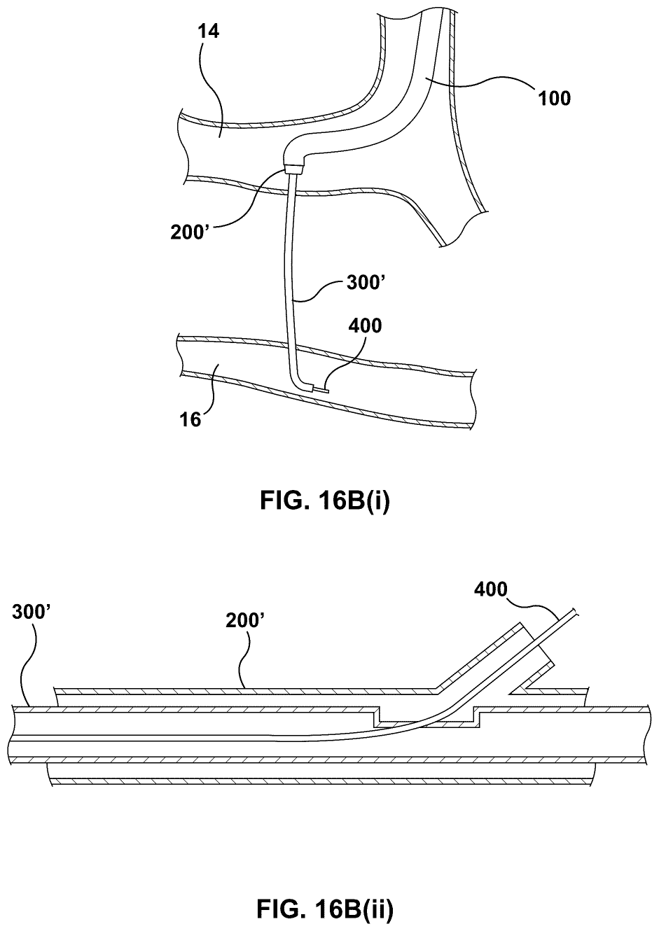

FIG. 15 shows a method for locking and advancing devices within a telescoping assembly during a TIPS procedure. At various stages of the procedure, certain devices need to remain stationary while others need to be advanced or retracted. The method may be better understood by referring also to FIG. 16A(i) to 16B(ii), which illustrate the devices in use during the procedure.

Method 1500 begins at step 1501, where a catheter 300′ comprising an elongate side opening is inserted into a dilator 200C comprising a side port and side opening, and the two side openings are aligned. Catheter may be an angled catheter or a straight catheter that is flexible enough to track into the portal vein.

At step 1502, the puncture wire 400 is inserted through the dilator 200C side opening, through the catheter 300′ side elongate opening, and extends out of the catheter 300 distal opening. The distal end of catheter 300′ may be located within the dilator 200c, such that the puncture wire 400 exists the dilator distal opening in a substantially straight configuration, as described herein.

At step 1503, the assembly is positioned in the hepatic vein, and the puncture wire is used to puncture through the liver. The puncture wire extends into the portal vein. At this stage the puncture wire may be unable to sufficiently track into the portal vein. FIG. 16A(i) shows the puncture wire 400 extending from the hepatic vein 14 to the portal vein 16. FIG. 16A(ii) shows a cross-section view of the proximal end of the assembly in a first position, described herein, where puncture wire 400 abuts a distal end of the catheter side elongate opening.

At step 1504, the puncture wire 400 is secured (locked) to the dilator 200′, using a locking feature associated with the dilator 200′. The catheter 300′ is not locked to the dilator or the puncture wire 400.

At step 1505, while the dilator and wire are locked, the catheter is advanced into the portal vein. An operator may hold the locked wire-dilator with one hand and advance the catheter with the other hand. Further, once the catheter has advanced sufficiently far over the puncture wire, the puncture wire curves so that it may track further into the portal vein. FIG. 16B(i) shows the catheter that has advanced over the puncture wire 400 and into the portal vein 16. FIG. 16B(ii) shows a cross-section of the proximal end of the assembly in a second position, described herein, where the puncture wire 400 abuts a proximal end of the side elongate opening and the catheter cannot be advanced any.

At step 1506, the puncture wire is unlocked from the dilator and advanced further into the portal vein. The puncture wire may then be used as a guidewire to continue with other steps of the TIPS procedure.

### Alternative Method for Advancing Devices During a Tips Procedure

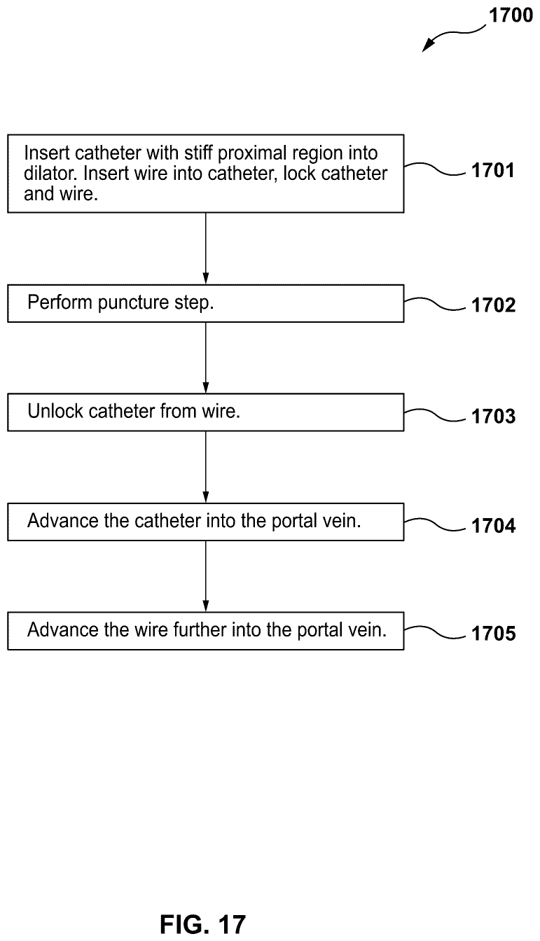

FIG. 17 shows an alternative method for locking and advancing devices within a telescoping assembly during a TIPS procedure. Method 1700 begins at step 1701, the wire is inserted into catheter, which is inserted into the dilator, which is inserted into the sheath, in a telescoping arrangement. All inner devices are inserted through the outer device proximal openings into the device primary lumens.

At step 1702, the puncture is performed. To perform a puncture through the liver, the wire and catheter are secured together, and the sheath and dilator are secured together. An operator may then hold one set of locked devices in each hand to perform the puncture. In some examples, the catheter and puncture wire are locked together in an arrangement such that the catheter distal end does not leave the dilator during the puncture. Alignment features may also be used to indicate the relative positions of the catheter and puncture wire, such that the catheter does not leave the dilator during the puncture.

At step 1703, after the puncture is complete, the puncture wire is in the portal vein. The catheter and wire are unlocked.

At step 1704, the catheter is advanced over the wire into the portal vein. Upon reaching the portal vein, the angle of the catheter curves, and the distal end of the puncture wire adopts a curve, and points along a direction of the portal vein.

Step 1704 may be performed in several ways as described herein. In one example, an operator may hold the sheath-dilator with a first hand, and also use fingers on the first hand to advance the catheter by gripping the stiff proximal region between the operators fingers. The operator holds the wire with the second hand during the advancement. In another example, the catheter may be advanced using an advancement mechanism described herein.

In another example, the wire may be secured to the sheath and/or dilator, using a locking feature described herein. The operator holds the secured sheath, dilator, and puncture wire with a first hand, and uses the second hand to advance the catheter.

At step 1705, after the catheter has advanced into the portal vein and the distal end of the puncture wire has adopted a curved configuration, the puncture wire is advanced further into the portal vein. If the puncture wire was locked in a previous step, it is unlocked at this step. The puncture wire may then be used as a guidewire to continue the TIPS procedure.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A telescoping assembly comprising:
a first elongate device, the first elongate device comprising a first device proximal region and defining a first device distal opening, the first elongate device further defining a first device lumen terminating at the first device distal opening, and a first device side opening in the first device proximal region in communication with the first device lumen; and
a second elongate device configured to be insertable into the first device lumen and moveable relative thereto, the second elongate device comprising a second device proximal region and defining a second device distal opening, the second elongate device further defining a second device lumen terminating at the second device distal opening, and a second device side opening in the second device proximal region in communication with the second device lumen;
wherein, the second elongate device is insertable into the first device lumen to an aligned configuration, whereby the first device side opening and the second device side opening are aligned to provide a passage from an external environment to the second device lumen through the first and second device side openings.

2. The telescoping assembly of claim 1, wherein at least one of the first device and second device comprises an alignment feature, for confirming when the first device and second device are in the aligned configuration.

3. The telescoping assembly of claim 1, wherein at least one of the first device and second device comprises a guiding feature for facilitating arrangement of the devices into the aligned configuration.

4. The telescoping assembly of claim 2, wherein the alignment feature comprises a longitudinal alignment feature.

5. The telescoping assembly of claim 2, wherein the alignment feature comprises a radial alignment feature.

6. The telescoping assembly of claim 1, wherein at least one of the first device and second device comprises a sealing feature for creating a seal between the first device side opening and the second device side opening.

7. The telescoping assembly of claim 1, wherein at least one of the first device and second device comprises a locking feature, whereby when the telescoping assembly is in a locked state, movement between the two is limited, and wherein the locking feature is configured to allow for transition between the locked state and an unlocked state.

8. The telescoping assembly of claim 7, wherein in the locked state, the first and second devices are in the aligned configuration.

9. The telescoping assembly of claim 1, wherein the second device lumen comprises a secondary lumen and wherein the second elongate device further defines a distal end opening and a primary lumen terminating at the distal end opening, the primary lumen having a larger diameter than the secondary lumen for receiving a third elongate device.

10. A method for interacting with fluid using a telescoping assembly, the telescoping assembly comprising a first elongate device and a second elongate device configured to be insertable into the first elongate device and moveable relative thereto, the first elongate device defining a first side opening and the second elongate device defining a second side opening, the method comprising the steps of:
interacting with fluid located in a lumen of the second elongate device through the first side opening;
withdrawing the second elongate device from the first elongate device; and
interacting with fluid located in a lumen of the first elongate device through the first side opening.

11. The method of claim 10, wherein, at least one of the steps of interacting with fluid located in a lumen of the second elongate device, or the step of interacting with fluid located in a lumen of the first elongate device, is selected from the group consisting of:
aspirating, injecting contrast, and measuring a pressure.

12. The method of claim 10 further comprising a step of inserting the second elongate device into the first elongate device.

13. The method of claim 12, wherein the method further comprises a step of aligning the second side opening with the first side opening.

14. The method of claim 10 further comprising a step of securing the second device to the first device.

15. A medical apparatus comprising:
a first elongate device comprising a first device proximal region and a first device distal region, the first elongate device defining a first device distal opening in the first device distal region and a first device proximal opening in the first device proximal region and a first device lumen extending therebetween;
a side port associated with the first device proximal region;
a second elongate device configured to be insertable into the first device lumen and moveable relative thereto, the second elongate device comprising a second device proximal region and a second device distal region, the second elongate device defining a second device distal opening in the second device distal region and a second device proximal opening in the second device proximal region and a second device lumen extending therebetween, the second device further comprising a sidewall in the second device proximal region, and further defining an elongated aperture in the sidewall; and
a third elongate device, configured to be insertable into the second lumen and moveable therethrough;
wherein the side port and elongated aperture are configured such that, when the first and second devices are in an aligned configuration, the third device is insertable through both the side port and elongated aperture into the second device lumen.

16. The medical apparatus of claim 15, wherein, while the third elongate device is positioned through the elongated aperture, longitudinal movement of the second elongate device relative to the first elongate device is limited substantially to a length of the elongate aperture.

17. The medical apparatus of claim 15 further comprising at least one locking feature, associated with the first elongate device, for restraining movement of the third elongate device relative to the first elongate device.

18. The medical apparatus of claim 15, wherein the first elongate device defines the side port.

19. The medical apparatus of claim 15, further comprising a multi-channel attachment connected to the first proximal opening, the multi-channel attachment defining the side port.

20. The medical apparatus of claim 15 further comprising a fourth device, the fourth device comprising a locking feature for prohibiting movement of the first device relative to the fourth device.
